(19) United States
(12) Patent Application Publication
(10) Pub. No.: US 2025/0100977 A1
(43) Pub. Date: Mar. 27, 2025

(54) MERCAPTOPYRIMIDINE COMPOUNDS THAT INHIBIT NONHOMOLOGOUS END JOINING AND METHODS THEREOF

(71) Applicants: INDIAN INSTITUTE OF SCIENCE, Bengaluru (IN); UNIVERSITY OF MYSORE, Manasagangothri, Mysore (IN)

(72) Inventors: Sathees C RAGHAVAN, Bengaluru (IN); Ujjayinee RAY, Bengaluru (IN); Vindya K GOPINATHA, Manasagangotri, Mysore (IN); Shivangi SHARMA, Bengaluru (IN); Kempegowda MANTELINGU, Manasagangotri, Mysore (IN); Kanchugarakoppal S RANGAPPA, Manasagangotri, Mysore (IN); Laijau GOYARY, Bangalore (IN)

(21) Appl. No.: 18/725,993

(22) PCT Filed: Dec. 30, 2022
(86) PCT No.: PCT/IN2022/051147

§ 371 Date: Jul. 1, 2024

(30) Foreign Application Priority Data
Dec. 31, 2021 (IN) 202141062306

(51) Int. Cl. C07D 239/56; A61K 31/505; A61K 31/506; A61K 31/513; A61K 31/7048; A61P 35/00; C07D 401/12; C07D 403/12; C07D 405/12; C07D 409/12; C07D 417/12

(52) U.S. Cl. CPC C07D 239/56; A61K 31/505; A61K 31/506; A61K 31/513; A61K 31/7048; A61P 35/00; C07D 401/12; C07D 403/12; C07D 405/12; C07D 409/12; C07D 417/12

(57) ABSTRACT

The present disclosure provides a compound of Formula I, its stereoisomers, intermediates, and pharmaceutically acceptable salts thereof, capable of inhibiting DNA Ligase IV enzyme activity and nonhomologous end joining (NHEJ). The present disclosure also provides a process for preparing the compounds of Formula I and methods thereof.

### FIELD OF INVENTION

The subject matter of the present disclosure broadly relates to mercaptopyrimidine compounds of Formula I, their stereoisomers, intermediates, and pharmaceutically acceptable salts thereof. The present disclosure further relates to the process of preparing the compounds of Formula (I).

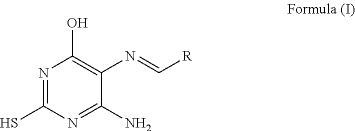

The compounds of the present disclosure inhibit the activity of DNA Ligase IV enzyme and the nonhomologous DNA end joining. The compounds of the present disclosure may be used in the treatment of cancers associated with high expression of DNA Ligase IV enzyme.

### BACKGROUND OF THE INVENTION

Clinically relevant small molecule inhibitors can sensitize cancer cells towards conventional therapies by aiming at the “Achilles' heel” in cancer, especially in chemo and radioresistant cancers (De Palma M and Hanahan D, Molecular oncology 6, 111-127, 2012; Lee Y T, European journal of pharmacology 834, 188-196, 2018; Sadar M D, Cancer research 71, 1208-1213,2011). Recent advancement in this field is to target the DNA repair mechanisms in cancer so as to result in unrepaired breaks and cellular apoptosis. Nonhomologous end joining (NHEJ) is one of the major DNA double-strand break repair pathways which get frequently deregulated in cancers such as, head and neck, pediatric, cervical, breast, lung, stomach and so on (Ray U and Raghavan S C, Biochemical Pharmacology, 182:114195, 2020; Begg A C et al., Nature Reviews Cancer 11, 239-253, 2011; Kuschel B et al., Human molecular genetics 11, 1399-1407, 2002; Srivastava M and Raghavan S C, Chemistry & biology 22, 17-29, 2015). The first protein of the pathway to bind to the break site was KU70/KU80 heterodimer. This was followed by involvement of DNA-PK and Artemis for end processing, strand synthesis by DNA polymerases μ/λ and finally ligation by DNA Ligase IV/XRCC4 (Lieber M, Journal of Biological Chemistry 293 (27): 10512-10523, 2010; Srivastava M and Raghavan S C, Chemistry & Biology 22, 17-29, 2015; Pannunzio N R et al., Journal of Biological Chemistry 293 (27): 10512-10523, 2018). Although these constitute the core factors of the NHEJ machinery, several recently discovered proteins such as PAXX, XLF, RNase H, etc serve as accessory factors for efficient repair of the double-strand breaks (Ghosh D and Raghavan S C, Trends in Genetics. 37 (6): 582-599, 2021; Ray U and Raghavan S C, DNA Repair (Amst). 106:103177, 2021).

However, no small molecule inhibitors of DNA double-strand break repair were taken to clinical settings. Most of the identified DNA-PK inhibitors (Wortmannin, NU-7026, NU-7441, etc) suffer from the limitations of nonspecificity with other kinases, or toxicity or poor bioavailability (Leahy J J et al., Bioorganic & medicinal chemistry letters 14, 6083-6087, 2004; Mohiuddin I S and Kang M H, Frontiers in oncology 9, 635,2019; Ray U and Raghavan S C, Oncogene, 39 (41): 6393-6405,2020), except M3814 which is currently in Phase II trial. KU70/KU80 inhibitors are still being investigated at in silico level (Weterings E et al., DNA Repair 43, 98-106, 2016). Although L189 was identified as Ligase IV inhibitor, it could target all three DNA ligases (Chen X et al., Cancer Research 68, 3169-3177, 2008; US20140113891A1). The first selective Ligase IV inhibitor with anticancer potential was identified as SCR7 (Srivastava et al., Cell 151, 1474-1487,2012; WO2014006518A1).

SCR7 has been utilised in several avenues of research, including biochemical inhibition of NHEJ, cancer therapy in cell lines and mice models, and genome editing across a multitude of model organisms. SCR7 is a small molecule inhibitor targeting NHEJ. SCR7 binds to the DNA binding domain (DBD) of Ligase IV, leading to the accumulation of unrepaired breaks and activation of apoptosis (Srivastava M et al., Cell 151, 1474-1487, 2012; Vartak S et al, The FEBS Journal 285, 3959-3976, 2018; Srivastava M and Raghavan S C, Chemistry & Biology 22, 17-29,2015). Treatment with SCR7 leads to cytotoxicity in cancer cells and tumour regression in multiple mouse models, as a single agent or when combined with radio and chemotherapy (Srivastava M et al., Cell 151, 1474-1487, 2012; Gopalakrishnan V et al., Molecular Carcinogenesis 60 (9): 627-643, 2021, Ray U and Raghavan S C, Biochemical Pharmacology, 182:114195, 2020).

Several research groups have used SCR7 as a biochemical inhibitor of Ligase IV for understanding the mechanism behind broken chromatin dynamics, chromosome territory relocation, protein recruitment in cell cycle phase dependent manner, drug screening, etc. (Reid et al., Nucleic Acids Research 45, 1872-1878, 2017; Kulashreshtha M et al., Nucleic Acids Research, 44 (17): 8272-91, 2016; Tripathi et al., Nature Communications 9, 1016, 2018; Yang et al., Oncotargetsand Therapy 11, 4945-4952,2018). Further, SCR7 has found remarkable application in precise genome editing using CRISPR-Cas in multiple model organisms (Maruyama et al., Nature Biotechnology 33, 538-542,2015; Chu et al., Nature Biotechnology 33, 543-548,2015; Singh et al., Genetics 199, 1-15,2015, Ray and Raghavan, Oncogene, 39 (41): 6393-6405,2020). Previous studies have also reported combination of SCR7 with the existing cancer treatment modalities such as, ionizing radiation, etoposide, doxorubicin, temozolomide, melphalan, oxaliplatin and 5-FU (Srivastava et al., Cell 151, 1474-1487,2012; Gopalakrishnan et al., Journal of Radiation and Cancer Research 9, 93-101,2018; Gopalakrishnan et al., Molecular Carcinogenesis60 (9): 627-643, 2021, Gkotzamanidou et al., Blood 128, 1214-1225, 2016; Kumar et al., 2019; Causse et al., Oncogene 38, 2767-2777, 2019).

Several forms of SCR7 have been identified and it was observed that parental SCR7 can undergo spontaneous cyclization to a more stable cyclized form, with the same molecular weight (334.09), molecular formula (C₁₈H₁₄N₄OS) and melting temperature (221-225° C.), which can get oxidized to SCR7-pyrazine (M.W. 332.07, molecular formula C₁₈H₁₂N₄OS, melting point 194-196° C.). SCR7-pyrazine shows nonspecific effects inside cells, although both forms can inhibit NHEJ in a Ligase IV-dependent manner (Vartak et al., The FEBS Journal 285, 3959-3976,2018). Not much advancement was made with the pluronic copolymer encapsulated SCR7, or water-soluble SCR7-pyrazine or water-soluble SCR7 (John F et al., Faraday Discussions, 177,2015; John F et al., Macromolecular Bioscience, 15 (4): 521-34, 2015; Ray et al., Clinical Oncology and Research, 2020; Pandey et al., Journal of Radiation and Cancer Research 10, 27-43,2019). The IC₅₀ values for the different forms of SCR7 were high in several cancer cell lines, particularly leukemia. Water-soluble SCR7-pyrazine inhibited all three DNA Ligases. These mainly posed as limitations for its clinical trial. Thus, there is a dire need for small molecule inhibitors specifically targeting NHEJ and Ligase IV at low doses.

### SUMMARY OF THE INVENTION

In an aspect of the present disclosure, there is provided a compound of Formula I,

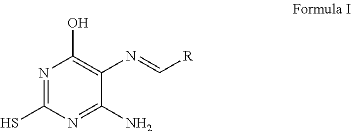

its stereoisomers, intermediates, and pharmaceutically acceptable salts thereof, wherein R is selected from a group consisting of substituted monocyclic C₆₋₁₀ aryl, C₁₋₁₂ alkyl, bicyclic C₈₋₁₂ aryl, C₃₋₁₀heteroaryl, C₃₋₁₀ cycloalkyl, and C₃₋₁₀heterocyclyl; wherein bicyclic C₈₋₁₂ aryl, C₁₋₁₂ alkyl, C₃₋₁₀heteroaryl, C₃₋₁₀ cycloalkyl, and C₃₋₁₀heterocyclyl are optionally substituted.

In second aspect of the present disclosure, there is provided a process for preparing the compound of Formula I, the process comprising: reacting 5,6-diamino-2-mercaptopyrimidin-4-ol with a compound of Formula B in the presence of an acid and a solvent under stirring for a time period of 6 to 8 hours to obtain the compound of Formula I,

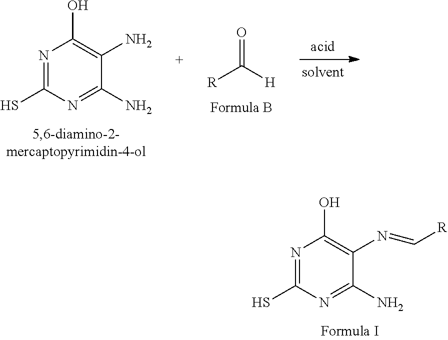

wherein R is selected from a group consisting of substituted monocyclic C₆₋₁₀ aryl, C₁₋₁₂ alkyl, bicyclic C₈₋₁₂ aryl, C₃₋₁₀ heteroaryl, C₃₋₁₀ cycloalkyl, and C₃₋₁₀heterocyclyl; wherein bicyclic C₈₋₁₂ aryl, C₁₋₁₂ alkyl, C₃₋₁₀ heteroaryl, C₃₋₁₀ cycloalkyl, and C₃₋₁₀ heterocyclyl are optionally substituted.

In third aspect of the present disclosure, there is provided a pharmaceutical composition comprising the compound of Formula I as disclosed herein, with a pharmaceutically acceptable adjuvant, carrier, or vehicle.

In fourth aspect of the present disclosure, there is provided a pharmaceutical combination comprising the compound of Formula I as disclosed herein, with one or more second therapeutic agent.

In fifth aspect of the present disclosure, there is provided a method of treating cancer in a subject in need thereof, comprising administering an effective amount of the compound of Formula I, or the pharmaceutical composition comprising the compound of Formula I with a pharmaceutically acceptable adjuvant, carrier, or vehicle, or the pharmaceutical combination comprising the compound of Formula I as disclosed herein, with one or more second therapeutic agent.

In sixth aspect of the present disclosure, there is provided a method for inhibiting DNA Ligase IV enzyme activity with an effective amount of the compound of Formula I, or the pharmaceutical composition comprising the compound of Formula I with a pharmaceutically acceptable adjuvant, carrier, or vehicle, or the pharmaceutical combination comprising the compound of Formula I with one or more second therapeutic agent.

In seventh aspect of the present disclosure, there is provided a method of inhibiting DNA double-strand break (DSB) through NHEJ, the method comprising contacting the compound of Formula I as disclosed herein with DNA Ligase.

In eighth aspect of the present disclosure, there is provided a use of a therapeutically effective amount of the compound of Formula I or the pharmaceutical composition comprising the compound of Formula I with a pharmaceutically acceptable adjuvant, carrier, or vehicle, or the pharmaceutical combination comprising the compound of Formula I as disclosed herein, with one or more second therapeutic agent, in the manufacture of a medicament for treating cancer, wherein the compound or the pharmaceutical composition or the combination is to be administered to a subject suffering from cancer.

In ninth aspect of the present disclosure, there is provided a DNA repair kit comprising the compound of Formula I or the pharmaceutical composition comprising the compound of Formula I with a pharmaceutically acceptable adjuvant, carrier, or vehicle, or the pharmaceutical combination comprising the compound of Formula I as disclosed herein, with one or more second therapeutic agent.

These and other features, aspects, and advantages of the present subject matter will be better understood with reference to the following description and the present disclosure. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE DRAWINGS

FIG. 1 (A, B and C) depicts the effect of compounds of Formula I on inhibition of end joining of rat tissue extracts. A. Representative denaturing PAGE profile for inhibition of end joining of rat testicular extracts in presence of compounds 01, 04, 05-07, 12, 13 (500 μM) and radiolabeled 5′-5′ compatible double-stranded DNA. Joined products were obtained at 150 nt position as dimers, and at 100 nt as circular products. B. Representative PAGE profile showing inhibition of end joining of rat testicular extracts by compounds 15-17 and 31-41 (500 μM). ‘M’ represents radiolabeled 50 bp DNA ladder, lane 1 denotes no protein control, and lanes 2, 3 denote joining in presence of equivalent DMSO, lane 11 denotes positive control (SCR7). C. Bar diagrams showing the quantification of extent of inhibition of each derivative (01-41) and SCR7 on end joining catalysed by rat testicular extract. Experiments were repeated at least thrice and quantifications were performed using Multi Gauge software. Error bars represent mean±SEM. Significance was calculated using ANOVA in GraphPad Prism (ns, not significant; *p<0.05, **p<0.005, ***p<0.0001),in accordance with an implementation of the present disclosure.

FIG. 2(A-D) depicts the effect of SCR7 derivatives on inhibition of end joining of purified Ligase IV/XRCC4. A. SDS-PAGE profile of DNA Ligase IV/XRCC4 overexpressed in bacteria and purified using Ni-NTA column chromatography. Protein was eluted using imidazole gradient. FT denotes flow through, W1 denotes wash, F1-F2 denotes elutions, ‘M’ denotes prestained protein ladder. B. Representative PAGE profile for inhibition of end joining assay using purified DNA Ligase IV/XRCC4 and SCR7 derivatives (01, 04, 05-07, 12, 13) at 200 μM concentration. Joined products were obtained at 45 nt position. SCR7 served as positive control. ‘M’ denotes 50 bp ladder. C. Representative PAGE profile for inhibition of end joining using DNA Ligase IV/XRCC4 and SCR7 derivatives 15-17 and 31-41 at 200 μM concentration. ‘M’ represents 50 bp ladder, Lane 1 denotes no protein control and lanes 2, 3 denote joining in presence of equivalent DMSO. D. Bar diagrams showing inhibition of end joining by 01-41 and SCR7. Experiments were repeated at least thrice and quantifications were performed using Multi Gauge software. Error bars represent mean±SEM. Significance was calculated using ANOVA in GraphPad Prism (ns, not significant; *p<0.05, **p<0.005, ***p<0.0001), in accordance with an implementation of the present disclosure.

FIG. 3(A-J) depicts effect of SCR7 derivatives on inhibition of end joining catalysed by purified Ligases I and III. A. SDS-PAGE profile of purified Ligase I from bacteria using size exclusion column. F1-F5 denotes column elutions, ‘M’ denotes prestained protein ladder. B-D. Representative PAGE profile for inhibition of end joining by purified Ligase I with compounds 01-13 (D), 15-17 (B) and 31-37 (C) at 200 μM concentration. ‘−’ and ‘+’ denote absence and presence of proteins or inhibitors in the reaction. ‘M’ denotes 50 bp marker. E. Bar diagram depicting quantification for end joining by Ligase I in presence of compounds 01-41 and SCR7.Experiments were repeated thrice and quantifications were performed using Multi Gauge software; ‘ns’ denote not significant. F. SDS-PAGE profile of purified DNA Ligase III/XRCC1 after Ni-NTA column elutions and ammonium sulphate precipitation. FT denotes flow through, W denotes wash, F1-F2 denotes column elutions. ‘M’ denotes prestained protein ladder. G-I. Representative PAGE profile for inhibition of end joining catalysed by purified Ligase III/XRCC1 with compounds, 01-13 (G), 15-17 (H) and 31-37 (I). J. Bar diagram depicting quantification for efficiency of end joining by 01-41 and SCR7. Experiments were repeated at least three times and quantifications were performed using Multi Gauge software. Error bars represent mean±SEM. Significance was calculated using ANOVA in GraphPad Prism (ns, not significant), in accordance with an implementation of the present disclosure.

FIG. 4(A-M) depicts the effect of SCR116 and SCR132 on end joining catalysed by cell-free extracts and purified proteins. A. Effect of increasing concentration of SCR116 (100, 200, 300, 400 and 500 μM) with rat testicular extracts. B. Bar diagram showing quantification for inhibition of end joining of rat testicular extracts with SCR116. C. Effect of increasing concentration of SCR132 (10, 50, 100, 200, 500 and 1000 μM) with rat testicular extracts. SCR7 (500 μM) was used as positive control. D. Bar diagram showing quantification for inhibition of end joining of rat testicular extracts with SCR132. E. Determination of specificity of SCR116 (1, 2, 5, 10, 50, 100, 200, 500, 1000 μM) with purified DNA Ligase IV/XRCC4. SCR7 (200 μM) served as positive control. F. Quantification showing inhibition of end joining catalysed by purified Ligase IV in presence of SCR116. G. Determination of specificity of SCR132 (10, 50, 100, 200, 500, 1000 μM) with purified Ligase IV/XRCC4. H. Quantification showing inhibition of end joining catalysed by purified Ligase IV in presence of SCR132. I. Representative PAGE profile showing complementation of purified Ligase IV/XRCC4 after incubation with testicular extracts in presence of SCR116 and SCR132. J. Quantification for end joining after complementation with Ligase IV/XRCC4 following treatment with SCR116 and SCR132 and testicular extract. Error bars denote mean±SEM. Significance was calculated from at least three independent experiments using GraphPad Prism (‘ns’ for non-significant, * for p<0.05, *** for p<0.005), K.Profile for purification of Ligase IV and XRCC4, L. Inhibition of end joining catalysed by purified Ligase III/XRCCI in presence of SCR116 and SCR132 (50, 100, 200, 500, 1000 μM). M. Inhibition of end joining catalysed by purified Ligase I in presence of increasing concentrations of SCR116 and SCR132 (10, 50, 100, 200, 500, 1000 μM), in accordance with an implementation of the present disclosure.

FIG. 5(A-I) depicts evaluation of IC₅₀ of SCR7 derivatives in Nalm6 and HeLa cell lines and molecular docking studies with DNA Ligase IV. A-B) Bar diagram showing percentage of viable cells after treatment with increasing concentration (0.1, 0.5, 1, 2, 5, 10 and 20 μM) of SCR116 (A) and SCR132 (B). C, D) Bar diagram showing cytotoxicity of increasing concentration (0.1, 0.5, 1, 2, 5, 10, 20 μM) of SCR116 and SCR132, respectively, in Nalm6. E, F) Bar diagram showing cytotoxicity of increasing concentration (0.1, 0.5, 1, 2, 5, 10, 20 μM) of SCR116 and SCR132, respectively, in Ligase IV knockout N114 cell line at 48 h. G) Docked pose of the top hit SCR117 with 3W5O. H) Docked pose of compound SCR116 with Ligase IV. I) Docked pose of compound SCR132 with Ligase IV, in accordance with an implementation of the present disclosure.

FIG. 6(A-C) depicts evaluation of efficiency of V(D)J recombination following treatment with SCR116 and SCR132. A. Schematic depicting steps involved in V(D)J recombination assay. B, C. depicts sequencing of different clones isolated from CA plates post transformation of pGG49 and pGG51, respectively. Arrows denote the cut sites. 12 and 23 RSS are depicted in golden and blue colours, in accordance with an implementation of the present disclosure.

FIG. 7(A-F) depicts evaluation of generation of double-strand breaks inside cells treated with compounds SCR116 and SCR132 alone or in combination, A. Immunofluorescence with 53BP1 in HeLa cells treated with increasing concentration of SCR116 (2 and 5 μM) and SCR132 (1 and 2 μM). Nucleus is stained in red using propidium iodide, while green signal represents 53BP1 foci (FITC). B. Quantification of 53BP1 foci inside HeLa cells treated with SCR116 and SCR132 for 24 h, as denoted using scatter plot. Significance was calculated using ANOVA in GraphPad Prism software (ns, not significant; *p<0.05, **p<0.005, ***p<0.0001). C-D. Bar graphs showing percentage of viable cells following treatment with the compounds alone, or in presence of γ-radiation (0.5 Gy) at 24 (C) and 48 h (D). 2 and 4 μM SCR116, and 1 and 2 μM SCR132 were used. E. Bar graph showing percentage of viable cells following treatment with SCR116 (0.375 μM) or SCR132 (0.125 μM) alone, or in presence of 0.25 μM etoposide at 48 h. F. Bar graph showing percentage of viable cells following treatment with SCR116 (0.375 μM) or SCR132 (0.125 μM) alone, or in presence of 0.375 μM 5-FU at 48 h, in accordance with an implementation of the present disclosure.

FIG. 8(A-F) depicts A. Representative images for HeLa cells after treatment with SCR116 and SCR132 for 24 h. B. Scatter plot depicting quantification of the percentage of DNA in the tail (bottom) and olive moment (top) in HeLa cells treated with SCR116 (5 μM) and SCR132 (2 μM) in presence of radiation (0.5 Gy). C, D. Representative images for Nalm6 cells (C) and N114 cells (D) following treatment with SCR116 and SCR132 for 24 h. E, F. Scatter plot depicting quantification of the percentage of 53BP1 foci treated with SCR116 (2, 5 μM) and SCR132 (1, 2 μM) in Nalm6 and N114, in accordance with an implementation of the present disclosure.

FIG. 9(A-H) depicts evaluation of mechanism of cell death caused by SCR116 and SCR132 on cancer cell lines and mouse tumor models, A. Western blot analysis of leukemic cell extracts from Nalm6 and Reh cells treated with increasing concentrations of SCR116 and SCR132 for 48 h. Ponceau stained blot was used as loading control B. Model depicting mechanism of cell death when treated with the inhibitors in leukemic cell lines. C. Scheme showing generation of mice allograft model for DLA/EAC tumor in mice thigh, followed by treatment with the compounds. D. Effect of SCR116 (10 mg/kg body weight) and SCR132 (5 mg/kg) on mice EAC tumor when administered intraperitoneally on alternate days. Black line denotes tumor control, maroon and blue lines denote treatment with compounds SCR116 and SCR132, respectively. E. Effect of SCR116 (3 mg/kg body weight) and SCR132 (5 mg/kg) on mice EAC tumor when administered intraperitoneally on alternate days. Black line denotes tumor control, while red (SCR116) and blue (SCR132) lines denote treatment with compounds. F. Histopathological analysis of mice EAC tumor tissues treated with SCR116 and SCR132. G-H. Analysis of body weight (G), blood parameters (G) and kidney-liver functions (H) in mice treated with intraperitoneal 6 doses of SCR116 (10 mg/kg) and SCR132 (5 mg/kg). Dark green, sea green and light green bar graphs denote vehicle control, SCR116 and SCR 132 treated animals. Significance is calculated using t-test, where ns denotes not significant and * denotes p<0.05, in accordance with an implementation of the present disclosure.

FIG. 10(A-T) depicts effects of oral doses of the compounds on tumor progression and bioavailability and effects on B and T cells, A-D. Pilot study to determine the effect of oral doses of SCR116 (10 mg/kg) and SCR132 (15 mg/kg) on alternate days on EAC (A-B) and DLA (C-D) mice tumor. Black line denotes tumor control treated with 0.1% methyl cellulose; green coloured lines denote treatment of EAC with SCR116 (A), pink coloured lines denote treatment of EAC with SCR132 (B). Red coloured lines denote treatment of DLA with SCR116 (C), blue coloured lines denote treatment of DLA with SCR132 (D). E-F. Effect of oral selected doses of SCR116 (10 mg/kg) and SCR132 (15 mg/kg) on mice allograft model, EAC (E), and DLA (F). G. Scheme for generation of mice xenograft model using cell lines, followed by treatment with oral doses of the compounds. H. Gross morphology of nude mice post-treatment with the compounds. I-J. Effect of 8 oral doses of SCR116 and SCR132 on Molt4 derived xenograft model. The black line denotes common tumor control; blue and orange lines denote SCR116 (I) and SCR132 (J), respectively. K-L. Effect of 8 oral doses of SCR116 and SCR132 on oral cancer, SCC131 (K) and SCC084 (L) derived xenograft model. Black line denotes common tumor control; blue and orange lines denote SCR116 and SCR132, respectively. M. Effect of the compounds on tumor progression of HCT116 derived xenograft model. N-P. HPLC profile of SCR116 (N) and SCR132 (O) in serum over time (0.5, 1, 2, 4, 6, 8, 10, 12, 24, 48 h) when orally administered in mice. AUC denotes maximum time when the compounds were actively present in serum, reflecting the bioavailability (P). Q. FACS profile of bone marrow of control and treated animals isolated 24 h after completion of 3 continuous doses of compounds. B-cells from bone marrow were stained with CD19-FITC. R. Quantifications of bone marrow (CD19+) and thymocytes (CD3+) numbers in control and treated cases after 21 days post treatment. T-cells from thymus were stained with CD3-FITC.S. Quantifications of bone marrow (CD19+) and thymocytes (CD3+) numbers in control and treated cases 24 h post treatment. T. Quantifications of bone marrow (CD19+) and thymocytes (CD3+) numbers in control and treated cases after 21 days post treatment, in accordance with an implementation of the present disclosure.

FIGS. 11(A, B and C) depicts the toxicity evaluation of the compounds in vivo, A. Effect on body weight in mice treated with a single oral dose of SCR116 and SCR132 (0, 50, 100, 200, 500 mg/kg). Graphs in shades of grey, red and violet denote vehicle control, SCR116 and SCR132, respectively. B. Effect of increasing doses of SCR116 (single dose of 50, 100, 200, 500 mg/kg) and SCR132 (single dose of 50, 100, 200, 400 mg/kg) on blood count in mice as analysed on 14th day. RBC denotes red blood corpuscles. MCHC denote mean corpuscular haemoglobin concentration, i.e. the average concentration of haemoglobin inside a single red blood cell. C. Effect of increasing doses of SCR116 and SCR132 on kidney liver function in mice as analysed on 14th day. BUN denotes blood urea nitrogen levels, while SGPT denote levels of serum glutamic pyruvic transaminase or alkaline transaminase, in accordance with an implementation of the present disclosure.

FIG. 12(A-B) depicts toxicity studies of SCR116 and SCR132 (A) Body weight analyses in male mice. (B) Body weight analyses in female mice, in accordance with an implementation of the present disclosure.

FIG. 13(A-E) depicts the blood parameters analysis treatment with SCR116 and SCR132. A. RBC (n=3). B. Hemoglobin (n=3). C. MCHC (n=3). D. Total leukocyte count (n=3). E. Platelet count (n=3), in accordance with an implementation of the present disclosure.

FIG. 14(A-B) depicts the evaluation of effect on B cells after SCR 116 treatment for 28 days in normal swiss albino mice A. After oral administration of low, medium, and high concentrations of SCR116 (80, 160 and 320 mg/kg b.wt. respectively). B is showing quantitation of the total B cell population (CD19+). (ns: not significant, *p<0.05: **p<0.005), in accordance with an implementation of the present disclosure.

FIG. 15(A-B) depicts the evaluation of the effect on T cells after SCR116 treatment for 28 days in normal swiss albino mice. A. After oral administration of low, medium and high concentrations of SCR116 (80, 160 and 320 mg/kg b.wt. respectively). B. is showing the quantitation of the total T cell population (CD3+). (ns: not significant, *p<0.05: **p<0.005), in accordance with an implementation of the present disclosure.

### DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to” “Including” and “including but not limited to” are used interchangeably.

In this specification, the prefix C_{x-y} as used in terms such as C_{x-y}alkyl and the like (where x and y are integers) indicates the numerical range of carbon atoms that are present in the group; for example, C₁₋₆alkyl includes C₁alkyl (methyl), C₂alkyl (ethyl), C₃alkyl (propyl and isopropyl) and C₄alkyl (butyl, 1-methylpropyl, 2-methylpropyl, and t-butyl). Unless specifically stated, the bonding atom of a group may be any suitable atom of that group; for example, propyl includes prop-1-yl and prop-2-yl.

The term “alkyl” refers to a monoradical branched or unbranched saturated hydrocarbon chain having from 1 to 6 carbon atoms. This term is exemplified by groups such as methyl, ethyl, n-propyl, isopropyl, n-butyl, iso-butyl, t-butyl, n-hexyl, and the like. The groups may be optionally substituted.

The term “aminoalkyl” refers to an amine group in which one or more hydrogen atoms are replaced by an alkyl group and hence the amine may be primary, secondary, or tertiary amino group. The point of attachment is the amine group. The term aminoalkyl is exemplified by groups such as —N(CH₃)₂, —NH(CH₃), and the like.

The term “cycloalkyl” refers to carbocyclic groups with 3 to 10 carbon atoms having a single cyclic ring or multiple condensed rings. Such cycloalkyl groups include, by way of example, single ring structures such as cyclopropyl, cyclobutyl, cyclopentyl, cyclopentenyl, cyclohexyl, and the like. The cycloalkyl groups may be saturated or partially saturated. The groups may be optionally substituted.

The terms “alkoxy” refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl. The alkoxy groups may be optionally substituted.

The term “aryl” refers to aromatic ring having a specified number of carbon atoms. For example, C₆₋₁₀ aryl refers to an aryl group having 6 to 10 carbon containing aromatic rings, or 6 member atoms. C₈₋₁₂ aryl refers to an aryl group having 8 to 12 carbon containing aryl groups. In the present disclosure, the aryl group is substituted with one or more groups as disclosed herein. The aryl group is monocyclic or bicyclic. Monocyclic aryl group is substituted with substituents as disclosed herein. The bicyclic group may be fused, bridged or spiral and is optionally substituted. Preferred aryl groups include, without limitation, phenyl, naphthalene and the like.

The term “Halo” or “Halogen”, alone or in combination with any other term means halogens such as chloro (Cl), fluoro (F), bromo (Br) and iodo (I).

The term “cyano” refers to —CN, the term “amino” refers to —NH₂, and the term “nitro” refers to —NO₂.

The term “heteroaryl” refers to aromatic rings containing from 1 to 3 heteroatoms selected from N, O and S in the ring. “Heteroaryl” groups may be optionally substituted with one or more substituents as defined herein. The “C₃₋₁₀ heteroaryl” rings having 3 to 10carbon as member atoms with one, two or three hetero atoms. Representative examples of heteroaryl group includes but not limited to thiazolyl, thiophenyl, imidazolyl, pyrazolyl, isoquinolinoyl, quinolinyl, pyroidinyl, furanyl, oxiranyl, oxetanyl, oxolanyl, oxanyl, furanyl, dioxanyl, pyranyl, aziridinyl, piperidinyl, tetrahydropyranyl, azepinyl, oxazepinyl and the like. Heteroaryl may be monocyclic, bicyclic or polycyclic, and may be bridged, spiral or fused rings.

The term “carbocyclyl” or “carbocycle” refers to a saturated, unsaturated ring having 3 to 10 ring atoms forming cyclic systems. Carbocyclic groups may be spiral or bridged systems, may be saturated or unsaturated. Carbocyclyl groups may be optionally substituted with one or more heteroatoms. Representative carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like

The term “heterocyclyl” refers to a “carbocyclyl” as defined herein, wherein one or more carbon atoms have been replaced with a heteroatom selected from O, N, or S. The heterocyclyl group may contain 3 to 10 carbon in the ring structure, substituted with one to three heteroatoms selected from O, N, S. The heterocyclyl may be saturated, unsaturated or partially saturated.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this disclosure, the heteroatoms such as nitrogen, oxygen or sulphur may have hydrogen substituents and/or any permissible substituents of organic compounds described herein which satisfy the valences of the heteroatoms.

The term “effective amount” means an amount of a compound or composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, the route of administration, and like factors within the knowledge and expertise of the attending physician.

The compounds described herein may contain one or more chiral centers and/or double bonds and therefore, may exist as stereoisomers, such as double-bond isomers (i.e., geometric isomers), regioisomers, enantiomers, or diastereomers. Accordingly, the chemical structures depicted herein encompass all possible enantiomers and stereoisomers of the illustrated or identified compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the person skilled in the art. The compounds may also exist in several tautomeric forms including the enol form, the keto form, and mixtures thereof. Accordingly, the chemical structures depicted herein encompass all possible tautomeric forms of the illustrated or identified compounds.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” embraces salts with a pharmaceutically acceptable acid or base. Pharmaceutically acceptable acids include both inorganic acids, for example hydrochloric, sulphuric, phosphoric, diphosphoric, hydrobromic, hydroiodic and nitric acid and organic acids, for example citric, fumaric, maleic, malic, mandelic, ascorbic, oxalic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases, for example alkyl amines, arylalkyl amines and heterocyclic amines.

One embodiment of the present disclosure embraces compounds of Formula I, and pharmaceutically acceptable salts thereof. Compounds of Formula I contain a basic functional group and are therefore capable of forming pharmaceutically acceptable acid addition salts by treatment with a suitable acid. Suitable acids include pharmaceutically acceptable inorganic acids and pharmaceutically acceptable organic acids. Representative pharmaceutically acceptable acid addition salts include hydrochloride, hydrobromide, nitrate, methylnitrate, sulfate, bisulfate, sulfamate, phosphate, acetate, hydroxyacetate, phenyl acetate, propionate, butyrate, iso-butyrate, valerate, maleate, hydroxymaleate, acrylate, fumarate, malate, tartrate, citrate, salicylate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, naphthoate, hydroxynaphthoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), aminobenzenesulfonate, p-toluenesulfonate (tosylate), and naphthalene-2-sulfonate.

The term “intermediate” refers to the compounds with same core structure of the compounds of the Formula I varying at specific allowed positions, for example alkyl positions.

The term “DNA Ligase IV” is a specific enzyme which facilitates joining of DNA double-strand breaks in organisms. DNA Ligase IV enzyme catalyzes the last step in the nonhomologous end joining pathway.

The term “NHEJ” is nonhomologous end joining is a pathway that repairs most of the double-strand breaks in human DNA, in which the break are ligated without a homologous template.

The term “adjuvant” refers to a substance or a compound which when added to a pharmaceutically active compound or a drug increases the efficacy or potency of the pharmaceutically active compound or the drug.

The term “carrier” refers to a substance which is used to carry and deliver the drug suitably. The carrier are preferably sterile liquids, such as water and oils, including those derived from petroleum, animal, vegetable, or synthetic sources, such as peanut oil, soybean oil, mineral oil, sesame oil, and the like. Physiological saline solutions and aqueous dextrose and glycerol solutions can also be used as liquid carriers

The term “vehicle” refers to a substance or a compound which may be a solvent that has little or no pharmacological activity used in the preparation of a pharmaceutical composition or a pharmaceutical combination.

The term “therapeutic agent” refers to a compound or a material which is used for a treatment of a condition or a disease or a disorder. In the present disclosure, the compound of Formula I can be used in combination with a (second) therapeutic agent for treatment of disease in specific cancers. The second therapeutic agent is selected from chemotherapeutic agent, radiotherapeutic agent, antiproliferative agent, antineoplastic agent, and DNA-damaging active compounds. These therapeutic agents are FDA approved agents used in accordance to the required pharmacological activity. The term radiotherapeutic agent refers to agents which exhibits radioactivity and the emitted radiation provides a therapeutic effect, for example ionizing radiation (γ-rays). The term chemotherapeutic agent refers to a compound or a substance which is used to treat cancers, representative examples includes but not limited to 5-fluorouracil, azacytidine, paclitaxel, bendamustine, etoposide, bleomycin, temozolomide, cisplatin, and AZD2461. The term antiproliferative agent refers to a compound or a substance which can inhibit the cell growth and is used for the treatment of cancer. The term antineoplastic agent refers to a compound or a substance which can inhibit or prevent the growth and spread of tumors or malignant cells and is used for the treatment of cancer. DNA-damaging active compounds are compounds which can induce DNA damage by causing structural changes in DNA.

A term once described, the same meaning applies for it, throughout the patent.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

As discussed, there is a need in the state of art for small molecule inhibitors of DNA Ligases, specifically DNA Ligase IV enzyme which in turn can inhibit NHEJ pathway for treating cancers. The known inhibitors of NHEJ are mostly those targeting DNA-PK involved in the initial steps of the repair process. Small molecule inhibitors of DNA-PK such as Wortmannin, NU-7026, NU-7441, etc exhibit nonspecific effects, toxicity and poor bioavailability in animal models. Further, inhibitors for KU70/KU80 heterodimer involved in binding and recognition of the break site, are limited with fewer in-silico lead generation recently. Among the inhibitors designed to target DNA Ligase IV, L189 is a pan-active ligase inhibitor targeting DNA Ligases I, III and IV, whereas SCR7 and its various forms exhibit high dosage in biochemical assay systems and in cancer cells (Chen X et al., Cancer Research 68, 3169-3177, 2008; US20140113891A1; Srivastava M et al., Cell 151, 1474-1487, 2012; WO2014006518A1; Vartak S et al, The FEBS Journal 285, 3959-3976, 2018; Srivastava M and Raghavan S C, Chemistry & Biology 22, 17-29,2015, Gopalakrishnan V et al., Molecular Carcinogenesis. 60 (9): 627-643, 2021, Ray U and Raghavan S C, Biochemical Pharmacology, 182:114195, 2020, Ray U and Raghavan S C, Oncogene, 39 (41): 6393-6405, 2020). Further, the oxidized version of SCR7 (pyrazine) and its water-soluble form exhibit non-specific effects ex vivo, and inhibit tumor progression in a pan-ligase manner. While the former works in pan-Ligase manner, SCR7 and its forms inhibit DNA Ligase IV at high concentrations (50% inhibition observed with 200 μM) and exhibit high maximal inhibitory concentration (IC₅₀) in cancer cell lines, particularly in leukemia where the IC₅₀>250 μM. The limitations of cancer treatment modalities such as surgery, radio and chemotherapy for cancer treatment have resulted in advancement of targeted therapy. In case of upregulated NHEJ repair, small molecule inhibitors can increase sensitivity of cancer cells to the common treatment modalities.

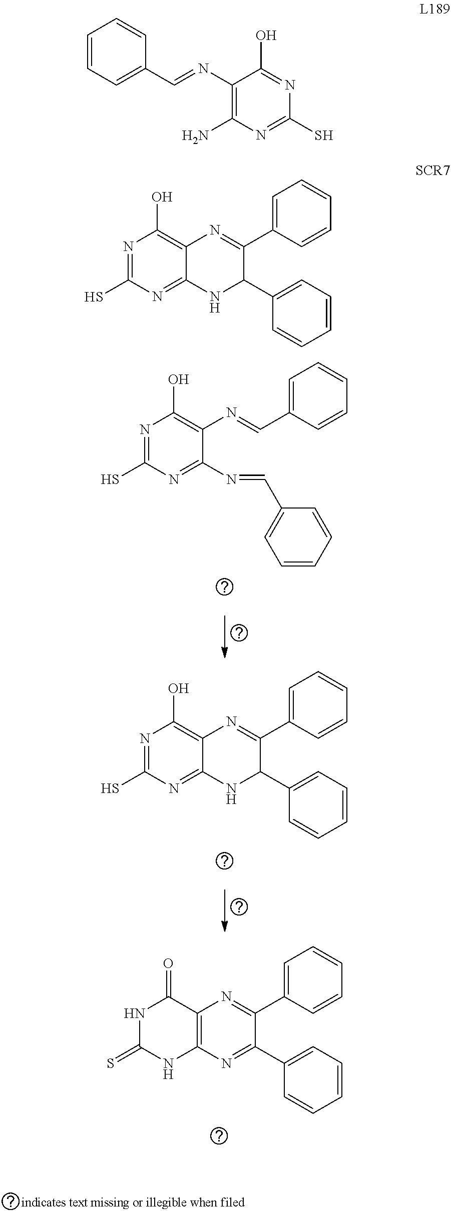

The selective inhibition is difficult to achieve and moreover the existing inhibitor compounds show high IC₅₀ values and higher toxicity. Therefore, the small molecule inhibitors of lower IC₅₀ values, higher bioavailability with appropriate toxicity are necessary for the compounds to exhibit better anti-cancer efficacy. Accordingly, the present disclosure provides mercaptopyrimidine compounds of Formula I which are small molecules and are inhibitors of DNA Ligase IV enzyme. The present disclosure also provides a process for preparing these compounds and their bioactivities towards cancer cell lines.

The present disclosure is not to be limited in scope by the specific embodiments described herein, which are intended for the purposes of exemplification only. Functionally-equivalent products, compositions, and methods are clearly within the scope of the disclosure, as described herein.

In an embodiment of the present disclosure, there is provided a compound of Formula I,

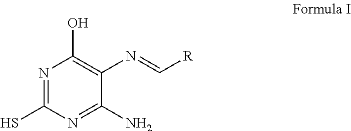

its stereoisomers, intermediates, and pharmaceutically acceptable salts thereof, wherein R is selected from a group consisting of substituted monocyclic C₆₋₁₀ aryl, C₁₋₁₂ alkyl, bicyclic C₈₋₁₂ aryl, C₃₋₁₀ heteroaryl, C₃₋₁₀ cycloalkyl, and C₃₋₁₀heterocyclyl; wherein bicyclic C₈₋₁₂ aryl, C₁₋₁₂ alkyl, C₃₋₁₀heteroaryl, C₃₋₁₀ cycloalkyl, and C₃₋₁₀heterocyclyl are optionally substituted.

In an embodiment of the present disclosure, there is provided a compound of Formula I, its stereoisomers, intermediates, and pharmaceutically acceptable salts thereof, wherein R is monocyclic C₆₋₁₀ aryl is substituted with one or more substituents selected from the group consisting of hydroxyl, —R′O(O)CR″, nitro, halogen, cyano, amino, amino C₁₋₆ alkyl, and C₁₋₆alkoxy; and R′ and R″ is independently selected from C₁₋₆alkyl.

In an embodiment of the present disclosure, there is provided a compound of Formula I, its stereoisomers, intermediates, and pharmaceutically acceptable salts thereof, wherein R is selected from C₁₋₁₂ alkyl, bicyclic C₈₋₁₂ aryl, C₃₋₁₀ cycloalkyl, C₃₋₁₀ heteroaryl or C₃₋₁₀heterocyclyl, wherein C₃₋₁₀ heteroaryl and C₃₋₁₀heterocyclyl has one to three hetero atoms selected from N, O and S; bicyclic C₈₋₁₂ aryl, C₃₋₁₀ cycloalkyl, C₃₋₁₀ heteroaryl, or C₃₋₁₀heterocyclyl is optionally substituted with one or more substituents selected from the group consisting of hydroxyl, —R′O(O)CR″, nitro, halogen, cyano, amino, aminoC₁₋₆alkyl, and C₁₋₆alkoxy; and R′ and R″ is independently selected from C₁₋₆alkyl.

In an embodiment of the present disclosure, there is provided a compound of Formula I, its stereoisomers, intermediates, and pharmaceutically acceptable salts thereof, wherein R is monocyclic substituted C₆₋₁₀ aryl, bicyclic C₈₋₁₂ aryl, or C₃₋₁₀ heteroaryl, wherein monocyclic C₆₋₁₀ aryl is substituted with one or more substituents selected from hydroxyl, halogen, aminoC₁₋₆alkyl, cyano, and C₁₋₆alkoxy; and bicyclic C₈₋₁₂ aryl, and C₃₋₁₀ heteroaryl is optionally substituted with one or more substituents selected from —CH₂O(O)CCH₃, nitro, halogen, and cyano.

In an embodiment of the present disclosure, there is provided a compound of Formula I, its stereoisomers, intermediates, and pharmaceutically acceptable salts thereof, wherein R is substituted monocyclic C₆₋₁₀ aryl, C₁₋₁₂ alkyl, bicyclic C₈₋₁₂ aryl, or C₃₋₁₀ heteroaryl, wherein monocyclic C₆₋₁₀ aryl is substituted with one or more substituents selected from hydroxyl, halogen, aminoC₁₋₂alkyl, cyano, and C₁₋₂alkoxy; and bicyclic C₈₋₁₂ aryl, and C₃₋₁₀ heteroaryl is optionally substituted with one or more substituents selected from —CH₂O(O)CCH₃, nitro, halogen, and cyano.

In an embodiment of the present disclosure, there is provided a compound of Formula I, its stereoisomers, intermediates, and pharmaceutically acceptable salts thereof, wherein R is C₃₋₁₀ heteroaryl having one to three atoms selected from N, O and S, substituted with one or more substituents selected from halogen, and nitro.

In an embodiment of the present disclosure, there is provided a compound of Formula I, wherein the compound is selected from the group consisting of:
i. (E)-6-amino-2-mercapto-5-(((5-nitrothiophen-2-yl)methylene)amino)pyrimidin-4-ol(16);
ii. (E)-6-amino-5-(((2,4-dichlorothiazol-5-yl)methylene)amino)-2-mercaptopyrimidin-4-ol (32);
iii. (E)-6-amino-2-mercapto-5-((thiophen-2-ylmethylene)amino)pyrimidin-4-ol (01);
iv. (E)-5-(((1H-imidazol-2-yl)methylene)amino)-6-amino-2-mercaptopyrimidin-4-ol (04);
v.6-amino-5-{[(E)-isoquinolin-4-ylmethylidene]amino}-2-mercaptopyrimidin-4-ol (05);
vi. (E)-6-amino-5-((2-hydroxybenzylidene)amino)-2-mercaptopyrimidin-4-ol (06);
vii. (E)-6-amino-2-mercapto-5-((pyridin-3-ylmethylene)amino)pyrimidin-4-ol (07);
viii. (E)-5-(((1H-pyrrol-2-yl)methylene)amino)-6-amino-2-mercaptopyrimidin-4-ol (12);
ix. (E)-6-amino-2-mercapto-5-((thiazol-2-ylmethylene)amino)pyrimidin-4-ol (13);
x. (E)-(5-(((4-amino-6-hydroxy-2-mercaptopyrimidin-5-yl)imino)methyl) furan-2-yl)methylacetate (15);
xi. (E)-6-amino-2-mercapto-5-((naphthalen-1-ylmethylene)amino)pyrimidin-4-ol (17);
xii. (E)-6-amino-5-(((2-chloropyridin-4-yl)methylene)amino)-2-mercaptopyrimidin-4-ol (31);
xiii. (E)-6-amino-5-(((3-fluoropyridin-4-yl)methylene)amino)-2-mercaptopyrimidin-4-ol (33);
xiv. (E)-5-(((4-amino-6-hydroxy-2-mercaptopyrimidin-5-yl)imino)methyl)thiophene-2-carbonitrile (34);
xv. (E)-6-amino-5-(((5-bromothiophen-2-yl)methylene)amino)-2-mercaptopyrimidin-4-ol (35);
xvi. (E)-4-(((4-amino-6-hydroxy-2-mercaptopyrimidin-5yl)imino)methyl)benzonitrile (36);
xvii. (E)-6-amino-5-((4-(dimethylamino)benzylidene)amino)-2-mercaptopyrimidin-4-ol (37);
xviii. (E)-6-amino-2-mercapto-5-((quinolin-2-ylmethylene)amino)pyrimidin-4-ol (38);
xix. (E)-6-amino-5-((4-hydroxy-3-methoxybenzylidene)amino)-2-mercaptopyrimidin-4-ol (39);
xx. (E)-6-amino-5-((5-bromo-2-hydroxybenzylidene)amino)-2-mercaptopyrimidin-4-ol (40); and
xxi. (E)-6-amino-5-((3,5-difluoro-4-hydroxybenzylidene)amino)-2-mercaptopyrimidin-4-ol (41).

In an embodiment of the present disclosure, there is provided a compound of Formula I, its stereoisomers, intermediates, and pharmaceutically acceptable salts thereof, wherein the compound inhibit DNA Ligase IV enzyme activity and nonhomologous end joining (NHEJ).

In an embodiment of the present disclosure, there is provided a compound of Formula I, its stereoisomers, intermediates, and pharmaceutically acceptable salts thereof, for use in the manufacture of a medicament for treatment of cancer associated with high expression of DNA Ligase IV enzyme.

In an embodiment of the present disclosure, there is provided a compound of Formula I, its stereoisomers, intermediates, and pharmaceutically acceptable salts thereof, for use in the manufacture of a medicament for treatment of cancer associated with expression of DNA Ligase IV enzyme.

In an embodiment of the present disclosure, there is provided a compound of Formula I, its stereoisomers, intermediates, and pharmaceutically acceptable salts thereof, for use in the treatment of cancer selected from but not limited to leukemia, lymphoma, head and neck carcinoma, colon carcinoma, lung cancer, prostate cancer and glioma.

In an embodiment of the present disclosure, there is provided a process for preparing process for preparing the compound of Formula I as disclosed herein, the process comprising: reacting 5,6-diamino-2-mercaptopyrimidin-4-ol with a compound of Formula B in the presence of an acid and a solvent under stirring for a time period of 6 to 8 hours to obtain the compound of Formula I,

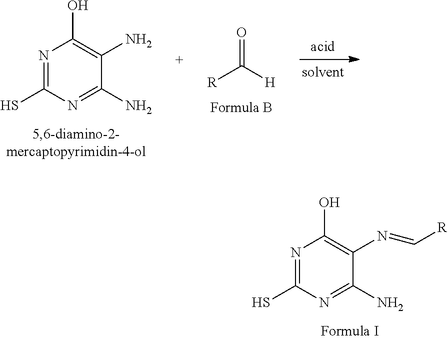

wherein R is selected from a group consisting of substituted monocyclic C₆₋₁₀ aryl, C₁₋₁₂ alkyl, bicyclic C₈₋₁₂ aryl, C₃₋₁₀ heteroaryl, C₃₋₁₀ cycloalkyl, and C₃₋₁₀heterocyclyl; wherein bicyclic C₈₋₁₂ aryl, C₃₋₁₀heteroaryl, C₃₋₁₀ cycloalkyl, and C₃₋₁₀heterocyclyl are optionally substituted.

In an embodiment of the present disclosure, there is provided a process for preparing the compound of Formula I as disclosed herein, wherein the acid is selected from glacial acetic acid, formic acid, or oxalic acid; and the solvent is dimethyl formamide or dimethyl sulfoxide. In another embodiment of the present disclosure, the acid is glacial acetic acid and the solvent is dimethyl formamide. In yet another embodiment of the present disclosure, 5,6-diamino-2-mercaptopyrimidin-4-ol and the compound of Formula B is taken in mole ratio of 1:1.

In an embodiment of the present disclosure, there is provided a pharmaceutical composition comprising the compound of Formula I, with a pharmaceutically acceptable adjuvant, carrier, or vehicle.

In an embodiment of the present disclosure, there is provided a pharmaceutical composition as disclosed herein, wherein the composition is in a form selected from a tablet, capsule, powder, syrup, solution, aerosol or suspension.

In an embodiment of the present disclosure, there is provided a pharmaceutical combination comprising the compound of Formula I with one or more second therapeutic agent.

In an embodiment of the present disclosure, there is provided a pharmaceutical combination comprising the compound of Formula I with one or more second therapeutic agent selected from chemotherapeutic agent, radiotherapeutic agent, antiproliferative agent, antineoplastic agent, DNA-damaging active compounds, or combinations thereof.

In an embodiment of the present disclosure, there is provided a pharmaceutical combination comprising the compound of Formula I with one or more chemotherapeutic agent selected from 5-fluorouracil, azacytidine, paclitaxel, bendamustine, etoposide, bleomycin, temozolomide, cisplatin, or AZD2461. In another embodiment of the present disclosure, the chemotherapeutic agent is 5-fluorouracil.

In an embodiment of the present disclosure, there is provided a pharmaceutical combination comprising the compound of Formula I with radiotherapeutic agent and the radiotherapeutic agent is an ionizing radiation, the γ-rays.

In an embodiment of the present disclosure, there is provided a method of treating cancer in a subject in need thereof, comprising administering an effective amount of the compound of Formula I, or the pharmaceutical composition comprising the compound of Formula I with a pharmaceutically acceptable adjuvant, carrier, or vehicle, or the pharmaceutical combination comprising the compound of Formula I with one or more second therapeutic agent.

In an embodiment of the present disclosure, there is provided a method of treating cancer in a subject in need thereof as disclosed herein, wherein the cancer is selected from leukemia, lymphoma, head and neck carcinoma, colon carcinoma, lung cancer, prostate cancer or glioma.

In an embodiment of the present disclosure, there is provided a method for inhibiting DNA Ligase IV enzyme activity with an effective amount of the compound of Formula I, or the pharmaceutical composition comprising the compound of Formula I with a pharmaceutically acceptable adjuvant, carrier, or vehicle, or the pharmaceutical combination comprising the compound of Formula I with one or more second therapeutic agent.

In an embodiment of the present disclosure, there is provided a method of inhibiting DNA double-strand break (DSB) through NHEJ, the method comprising contacting the compound of Formula I with DNA Ligase IV.

In an embodiment of the present disclosure, there is provided a method of inhibiting DNA double-strand break (DSB) through NHEJ, the method comprising contacting the compound of Formula I with DNA Ligase IV enzyme and the method is carried out by NHEJ.

In an embodiment of the present disclosure, there is provided a use of a therapeutically effective amount of the compound of Formula I or the pharmaceutical composition comprising the compound of Formula I with a pharmaceutically acceptable adjuvant, carrier, or vehicle, or the pharmaceutical combination comprising the compound of Formula I with one or more second therapeutic agent, in the manufacture of a medicament for treating cancer, wherein the compound or the pharmaceutical composition or the combination is to be administered to a subject suffering from cancer.

In an embodiment of the present disclosure, there is provided a DNA repair kit comprising the compound of Formula I, or the pharmaceutical composition comprising the compound of Formula I with a pharmaceutically acceptable adjuvant, carrier, or vehicle, or the pharmaceutical combination comprising the compound of Formula I with one or more second therapeutic agent.

In an embodiment of the present disclosure, there is provided a compound of Formula I, or the pharmaceutical composition comprising the compound of Formula I with a pharmaceutically acceptable adjuvant, carrier, or vehicle, or the pharmaceutical combination comprising the compound of Formula I with one or more second therapeutic agent which can be administered to a subject in need thereof through intraperitoneal or oral routes.

“Combination therapy” includes the administration of the subject compounds in further combination with other biologically active ingredients (such as, but are not limited to, different antineoplastic agent) and non-drug therapies (such as, but are not limited to, surgery or radiation treatment). The compounds described herein can be used in combination with other pharmaceutically active compounds, preferably, which will enhance the effect of the compounds of the invention. The compounds can be administered simultaneously or sequentially to the other drug therapy.

Although the subject matter has been described in considerable detail with reference to certain examples and implementations thereof, other implementations are possible.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices, and materials are described herein. It is to be understood that this disclosure is not limited to particular methods, and experimental conditions described, as such methods and conditions may apply.

The following examples provide the details about the synthesis, and bioactivities of the compounds of the present disclosure. It should be understood the following is representative only, and that the invention is not limited by the details set forth in these examples.

The compounds of the invention may be made by a variety of methods, including standard chemistry. Any previously defined variable will continue to have the previously defined meaning unless otherwise indicated. Illustrative general synthetic methods are set out in the following schemes, and can be readily adapted to prepare other compounds of the invention.

There is provided a process as shown in the following schemel, for the preparation of compounds of the Formula I, wherein all the groups are as defined earlier.

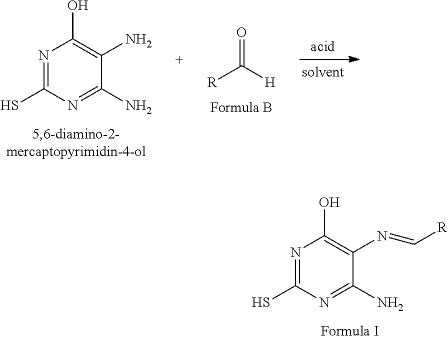

wherein R is selected from a group consisting of substituted monocyclic C₆₋₁₀ aryl, C₁₋₁₂ alkyl, bicyclic C₈₋₁₂ aryl, C₃₋₁₀ heteroaryl, C₃₋₁₀ cycloalkyl, and C₃₋₁₀ heterocyclyl; and bicyclic C₈₋₁₂ aryl, C₁₋₁₂ alkyl, C₃₋₁₀ heteroaryl, C₃₋₁₀ cycloalkyl, and C₃₋₁₀heterocyclyl are optionally substituted.

The compounds of the present disclosure were synthesized from 5,6-diamino-2-mercaptopyrimidin-4-ol with their corresponding heterocyclic aldehydes using glacial acetic acid (0.5 ml) in DMF (dimethyl formamide) as solvent. Structures were confirmed by 1H NMR, 13C NMR and mass spectrometric analyses.

The prepared compounds were characterized by spectroscopic techniques ¹H, ¹³C NMR and LC-MS. All compounds of the present invention showed similar spectroscopic data with strong characteristic peaks in ¹H NMR. —N═C—H resonates between 9-10 ppm range as a singlet, which confirmed the formation of Schiff base, aromatic protons resonate between 6.5-7.5 ppm range as a multiplets, —NH₂ resonates as a broad singlet between 6.5-7 ppm, whereas highly exchangeable protons —OH and —SH protons resonates between 11-12 ppm as a singlet. ¹³C NMR chemical shifts for compounds were observed in their expected region. The mass spectra of all compounds exhibit well defined molecular ions.

### Synthesis of (E)-6-amino-2-mercapto-5-(((5-nitrothiophen-2-yl)methylene)amino)pyrimidin-4-ol (SCR116 or 16)

Mixture of 5,6-diamino-2-mercaptopyrimidin-4-ol (0.005 mol), 5-nitrothiophene-2-carbaldehyde (0.005 mol) and glacial acetic acid (0.5 ml) was taken in a DMF and stirred for 6-8 hours at RT. After completion of the reaction, the reaction mixture was poured into ice cold water, the solid obtained was filtered and recrystallized from DMF-ethanol mixture to obtain (E)-6-amino-2-mercapto-5-(((5-nitrothiophen-2-yl)methylene)amino)pyrimidin-4-ol (SCR116 or 16). Brown solid; Yield: 92%; Melting Point: >300° C.; ¹H NMR (DMSO-d₆, 400 MHZ): δ=12.10 (s, 1H), 11.92 (Br, 1H), 9.70 (s, 1H), 8.06 (d, J=4 Hz, 1H), 7.50 (d, J=4 Hz, 1H), 6.40-7.20 (Br, 2H);13C NMR (DMSO-d6, 100 MHz): δ=172.8, 157.4, 153.6, 152.9, 150.9, 143.3, 131.2, 128.2, 102.4; LCMS (ESI) [M−H]⁻ calculated C₉H₇N₅O₃S: 2295.9990 found 295.9027.

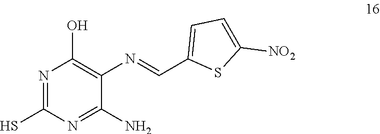 [SMILES: Nc1nc(S)nc(O)c1/N=C/c1ccc([N+](=O)[O-])s1]

### Synthesis of (E)-6-amino-5-(((2,4-dichlorothiazol-5-yl)methylene)amino)-2-mercaptopyrimidin-4-ol (SCR132 or 32)

Mixture of 5,6-diamino-2-mercaptopyrimidin-4-ol (0.005 mol), 2,4-dichlorothiazole-5-carbaldehyde (0.005 mol) and glacial acetic acid (0.5 ml) was taken in a DMF and stirred for 6-8 hours at RT. After completion of the reaction, the reaction mixture was poured into ice cold water, the solid obtained was filtered and washed with ethanol to obtain (E)-6-amino-5-(((2,4-dichlorothiazol-5-yl)methylene)amino)-2-mercaptopyrimidin-4-ol (SCR132 or 32). Yellow solid; Yield: 88%; Melting Point: >300° C.; ¹H NMR (DMSO-d₆, 400 MHZ): δ=12.14 (s, 1H), 11.93 (s, 1H), 9.63 (s, 1H); LCMS (ESI) [M−H]⁻ calculated C₈H₅Cl₂N₅OS₂: 319.9313 found 319.8349.

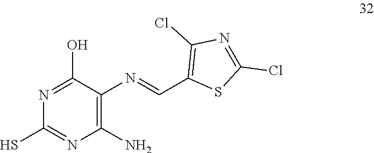 [SMILES: Nc1nc(S)nc(O)c1/N=C/c1sc(Cl)nc1Cl]

Similarly other compounds using the reaction scheme were prepared. The scheme involved condensation of 5,6-diamino-2-mercaptopyrimidin-4-ol with different heterocyclic aldehydes and substituted aromatic benzaldehydes in DMF with corresponding molar ratio of 1:1, to generate the compounds of Formula I.

(E)-6-amino-2-mercapto-5-((thiophen-2-ylmethylene)amino)pyrimidin-4-ol (01): Pale yellow solid; Yield: 79%; Melting Point: 268-270° C.; ¹H NMR (DMSO-d₆, 400 MHZ): δ=11.99 (s, 1H), 11.860 (s, 1H), 9.74 (s, 1H), 7.61 (d, J=4.8 Hz, 1H), 7.44 (d, J=3.2 Hz, 1H), 7.10 (dd, J=4.8 Hz, 3.6 Hz, 1H), 6.41 (s, 2H); ¹³C NMR (DMSO-d₆, 100 MHz): δ=172.1, 157.5, 151.9, 146.4, 145.6, 130.0, 129.0, 128.4, 102.2; LCMS (ESI): [M+H]⁺ calculated C₉H₈N₄OS₂: 253.0173 found 253.4871.

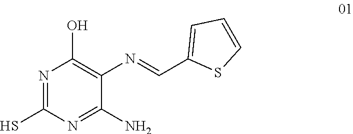 [SMILES: Nc1nc(S)nc(O)c1/N=C/c1cccs1]

(E)-5-(((1H-imidazol-2-yl)methylene)amino)-6-amino-2-mercaptopyrimidin-4-ol (04): Pale yellow solid; Yield 85%; Melting Point: 290-292° C.; ¹H NMR (DMSO-d₆, 400 MHz): δ=12.49 (Br, 1H), 12.05 (Br, 1H), 11.94 (s, 1H), 9.36 (s, 1H), 7.17 (s, 2H), 6.48 (Br, 2H); ¹³C NMR (DMSO-d₆, 100 MHz): δ=172.5, 157.4, 152.8, 147.9, 141.0, 101.7; LCMS (ESI): [M+H]⁺ calculated C₈H₈N₆OS: 237.0514 found 237.5187.

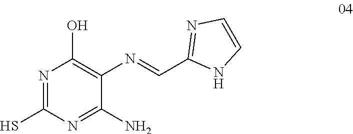 [SMILES: Nc1nc(S)nc(O)c1/N=C/c1ncc[nH]1]

6-amino-5-{[(E)-isoquinolin-4-ylmethylidene]amino}-2-mercaptopyrimidin-4-ol (05): Orange solid; Yield 89%; Melting Point: 245-247° C.; ¹H NMR (DMSO-d₆, 400 MHz): δ=12.05 (Br, 2H), 10.39 (s, 1H), 9.28 (s, 1H), 9.21 (s, 1H), 8.47 (d, J=8.4 Hz, 1H), 8.16 (d, J=8 Hz, 1H), 7.85-7.89 (m, 1H), 7.69-7.72 (m, 1H), 6.86 (Br, 2H); ¹³C NMR (DMSO-d₆, 100 MHz): δ=172.5, 157.5, 153.1, 152.9, 147.7, 142.3, 133.2.132.1, 128.9, 128.3, 127.9, 126.8, 122.5, 103.2; LCMS (ESI): [M+H]⁺ calculated C₁₄H₁₁N₅OS: 298.0718 found 298.7006.

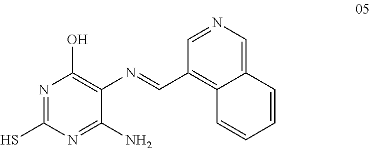 [SMILES: Nc1nc(S)nc(O)c1/N=C/c1cncc2ccccc12]

(E)-6-amino-5-((2-hydroxybenzylidene)amino)-2-mercaptopyrimidin-4-ol (06): Yellow solid; Yield: 82%; Melting Point: 278-280° C.; ¹H NMR (DMSO-d₆, 400 MHz): δ=12.04 (s, 1H), 11.83 (s, 1H), 11.41 (s, 1H), 9.72 (s, 1H), 7.63 (d, J=7.2 Hz, 1H), 7.20-7.23 (m, 1H), 6.82-6.85 (m, 2H), 6.54 (Br, 2H); ¹³C NMR (DMSO-d₆, 100 MHz): δ=172.1, 158.6, 157.5, 155.4, 151.2, 131.6, 130.0, 122.3, 119.5, 116.5, 102.5; LCMS (ESI): [M+H]⁺ calculated: C₁₁H₁₀N₄O₂S 263.0558 found 263.5534.

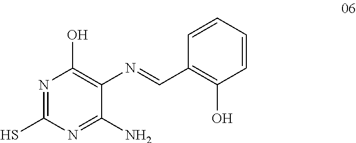 [SMILES: Nc1nc(S)nc(O)c1/N=C/c1ccccc1O]

(E)-6-amino-2-mercapto-5-((pyridin-3-ylmethylene)amino)pyrimidin-4-ol (07): Yellow solid; Yield: 90%; Melting Point: 286-288° C.; ¹H NMR (DMSO-d₆, 400 MHZ): δ=12.00 (s, 1H), 11.85 (s, 1H), 9.67 (s, 1H), 9.01 (s, 1H), 8.53 (d, J=4 Hz, 1H), 8.30 (dd, J=6.2 Hz, 1.8 Hz, 1H), 7.39-7.42 (m, 1H), 6.84 (Br, 2H); ¹³C NMR (DMSO-d₆, 100 MHz): δ=172.5, 157.4, 152.9, 150.4, 149.6, 148.8, 134.3, 134.1, 124.1, 102.4; LCMS (ESI): [M+H]⁺ calculated C₁₀H₉N₅OS: 248.0561 found 248.5344.

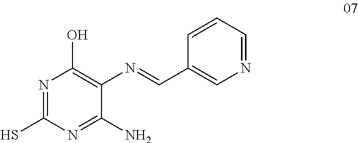 [SMILES: Nc1nc(S)nc(O)c1/N=C/c1cccnc1]

(E)-5-(((1H-pyrrol-2-yl)methylene)amino)-6-amino-2-mercaptopyrimidin-4-ol (12): Green solid; Yield: 86%; Melting Point: 278-280° C.; 1H NMR (DMSO-d₆, 400 MHZ): δ=11.91 (s, 1H), 11.85 (s, 1H), 11.41 (s, 1H), 9.35 (s, 1H), 6.97 (s, 1H), 6.39 (s, 1H), 6.08-6.10 (m, 1H), 6.80 (Br, 2H); ¹³C NMR

(DMSO-d₆, 100 MHz): δ=171.6, 157.5, 151.9, 142.8, 133.2, 121.9, 113.0, 109.6, 102.1; LCMS (ESI): [M+H]⁺ calculated C₉H₉N₅OS: 236.0561 found 236.5211.

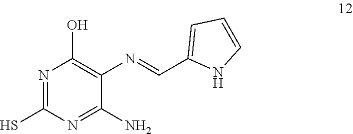 [SMILES: Nc1nc(S)nc(O)c1/N=C/c1ccc[nH]1]

(E)-6-amino-2-mercapto-5-((thiazol-2-ylmethylene)amino)pyrimidin-4-ol (13): Yellow solid; Yield: 80%; Melting Point: 268-270° C.; ¹H NMR (DMSO-d₆, 400 MHZ): δ=12.11 (s, 1H), 11.95 (Br, 1H), 9.71 (s, 1H), 7.88 (d, J=3.2 Hz, 1H), 7.74 (d, J=2.8 Hz, 1H), 6.68 (Br, 2H); ¹³C NMR (DMSO-d₆, 100 MHz): δ=172.8, 169.9, 157.5, 152.7, 144.9, 144.5, 122.1, 102.0; LCMS (ESI): [M+H]⁺ calculated C₈H₇N₅OS₂: 254.0050 found 254.4989.

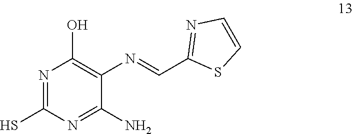 [SMILES: Nc1nc(S)nc(O)c1/N=C/c1nccs1]

(E)-(5-(((4-amino-6-hydroxy-2-mercaptopyrimidin-5-yl)imino)methyl)furan-2-yl)methylacetate (15): Brown solid; Yield: 82%; Melting Point: 195-197° C.; ¹H NMR (DMSO-d₆, 400 MHZ): δ=11.99 (s, 1H), 11.87 (s, 1H), 9.45 (s, 1H), 6.97 (d, J=3.6 Hz, 1H), 6.62 (d, J=3.6 Hz, 1H), 6.55 (Br, 2H), 5.04 (s, 2H), 2.03 (s, 3H); ¹³C NMR (DMSO-d₆, 100 MHz): δ=172.2, 170.4, 157.4, 154.7, 152.3, 151.1, 140.7, 113.5, 112.8, 102.4, 58.1, 21.0; LCMS (ESI): [M+H]⁺ calculated C₁₂H₁₂N₄O₄S: 309.0613 found 309.6033.

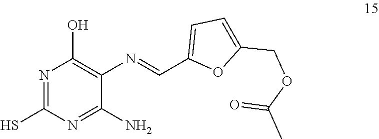 [SMILES: CC(=O)OCc1ccc(/C=N/c2c(N)nc(S)nc2O)o1]

(E)-6-amino-2-mercapto-5-((naphthalen-1-ylmethylene)amino)pyrimidin-4-ol (17): Yellow solid; Yield: 83%; Melting Point: 266-268° C.; ¹H NMR (DMSO-d₆, 400 MHz): δ=12.01 (Br, 2H), 10.54 (s, 1H), 8.37-8.46 (m, 2H), 7.94-8.07 (m, 2H), 7.52-7.63 (m, 3H), 6.75 (Br, 2H); ¹³C NMR (DMSO-d₆, 100 MHz): δ=172.3, 157.6, 152.7, 149,4, 133.9, 133.4, 131.4, 130.2, 129.2, 127.3, 126.0, 125.2, 122.9, 103.3; LCMS (ESI): [M+H]⁺ calculated C₁₅H₁₂N₄OS: 297.0765 found 297.5886.

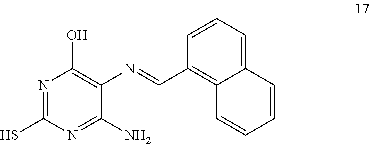 [SMILES: Nc1nc(S)nc(O)c1/N=C/c1cccc2ccccc12]

(E)-6-amino-5-(((2-chloropyridin-4-yl)methylene)amino)-2-mercaptopyrimidin-4-ol (31): Yellow solid; Yield: 86%; Melting Point: 276-278° C.; ¹H NMR (DMSO-d₆, 400 MHZ): δ=12.06 (s, 1H), 11.92 (Br, 1H), 9.91 (s, 1H), 8.76-8.78 (m, 1H), 8.33-8.35 (m, 1H), 7.41-7.45 (m, 1H), 6.60 (Br, 2H); ¹³C NMR (DMSO-d₆, 100 MHz): δ=172.7, 157.4, 153.2, 150.2, 145.5, 136.7, 132.2, 123.8, 102.9; LCMS (ESI): [M+H]⁺ calculated C₁₀H₈ClN₅OS: 282.0109 found 282.3125.

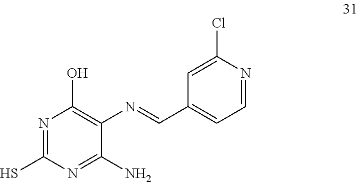 [SMILES: Nc1nc(S)nc(O)c1/N=C/c1ccnc(Cl)c1]

(E)-6-amino-5-(((3-fluoropyridin-4-yl)methylene)amino)-2-mercaptopyrimidin-4-ol (33) Yellow solid; Yield: 88%; Melting Point: 264-266° C.; ¹H NMR (DMSO-d₆, 400 MHZ): δ=12.11 (s, 1H), 11.96 (Br, 1H), 9.79 (s, 1H), 8.55 (s, 1H), 8.40 (d, J=4.8 Hz, 1H), 8.26-8.29 (m, 1H), 6.60 (Br, 2H); ¹³C NMR (DMSO-d₆, 100 MHz): δ=173.0, 157.4, 153.6, 146.1, 140.3, 139.1, 138.8, 132.7, 120.8, 102.8; LCMS (ESI): [M+H]⁺ calculated C₁₀H₈FN₅OS: 266.0467 found 266.5472.

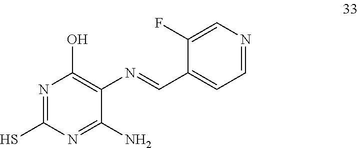 [SMILES: Nc1nc(S)nc(O)c1/N=C/c1ccncc1F]

(E)-5-(((4-amino-6-hydroxy-2-mercaptopyrimidin-5-yl)imino)methyl)thiophene-2-carbonitrile (34): Yellow solid; Yield: 88%; Melting Point: >300° C.; ¹H NMR (DMSO-d₆, 400 MHZ): δ=12.07 (s, 1H), 11.93 (Br, 1H), 9.71 (s, 1H), 7.86 (d, J=4 Hz, 1H), 7.52 (d, J=3.6 Hz, 1H), 6.65 (Br, 2H); ¹³C NMR (DMSO-d₆, 100 MHz): δ=172.6, 157.5, 153.2, 152.7, 143.5, 139.9, 129.0, 115.2, 108.5, 102.1; LCMS (ESI): [M+H]⁺ calculated C₁₀H₇N₅OS₂: 278.0126 found 278.5233.

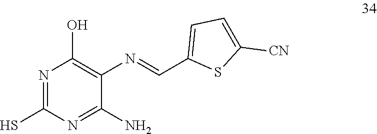 [SMILES: N#Cc1ccc(/C=N/c2c(N)nc(S)nc2O)s1]

(E)-6-amino-5-(((5-bromothiophen-2-yl)methylene)amino)-2-mercaptopyrimidin-4-ol (35): Yellow solid; Yield: 82%; Melting Point: 230-232° C.; ¹H NMR (DMSO-d₆, 400 MHz): δ=11.99 (s, 1H), 11.86 (Br, 1H), 9.62 (s, 1H), 7.23 (d, J=4 Hz, 1H), 7.17 (d, J=3.6 Hz, 1H), 6.47 (Br, 2H); ¹³C NMR (DMSO-d₆, 100 MHz): δ=172.2, 157.5, 152.1, 147.7, 145.2, 131.7, 130.2, 115.1, 102.0; LCMS (ESI): [M+H]⁺ calculated C₉H₇BrN₄OS₂: 333.9224 found 334.0245.

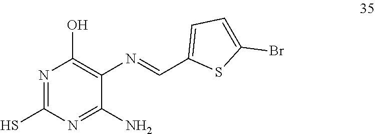 [SMILES: Nc1nc(S)nc(O)c1/N=C/c1ccc(Br)s1]

(E)-4-(((4-amino-6-hydroxy-2-mercaptopyrimidin-5yl)imino)methyl)benzonitrile (36): Pale yellow solid; Yield: 85%; Melting Point: 280-282° C.; ¹H NMR (DMSO-d₆, 400 MHZ): δ=12.02 (s, 1H), 11.86 (Br, 1H), 9.63 (s, 1H), 8.52 (s, 1H), 8.05 (d, J=7.6 Hz, 1H), 7.75 (d, J=7.6 Hz, 1H), 7.54-7.58 (m, 1H), 6.50 (Br, 2H), ¹³C NMR (DMSO-d₆, 100 MHz): δ=172.1, 156.9, 152.6, 148.6, 139.4, 132.4,132.3, 130.3, 129.6, 118.8, 117.7, 101.7; LCMS (ESI): [M+H]⁺ calculated C₁₂H₉N₅OS: 272.0561found 272.5565.

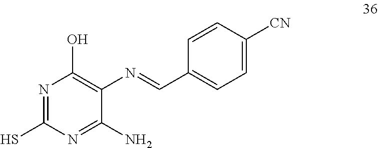 [SMILES: N#Cc1ccc(/C=N/c2c(N)nc(S)nc2O)cc1]

(E)-6-amino-5-((4-(dimethylamino)benzylidene)amino)-2-mercaptopyrimidin-4-ol (37): Yellow solid; Yield: 83%; Melting Point: 240-242° C.; ¹H NMR (DMSO-d₆, 400 MHZ): δ=11.86 (Br, 2H), 9.49 (s, 1H), 7.65 (d, J=8.8 Hz, 2H), 6.66 (d, J=8.8 Hz, 2H), 6.50 (Br, 2H), 2.92 (s, 6H); ¹³C NMR (DMSO-d₆, 100 MHz): δ=171.5, 157.5, 153.1, 151.8, 151.7, 129.3, 126.5, 112.0, 102.9; LCMS (ESI): [M+H]⁺ calculated C₁₃H₁₅N₅OS: 290.1031 found 290.6207.

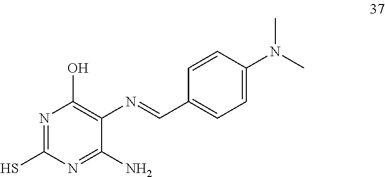 [SMILES: CN(C)c1ccc(/C=N/c2c(N)nc(S)nc2O)cc1]

(E)-6-amino-2-mercapto-5-((quinolin-2-ylmethylene)amino)pyrimidin-4-ol (38) Brown solid; Yield: 83%; Melting Point: 222-224° C.; ¹H NMR (DMSO-d₆, 400 MHz): δ=12.08 (s, 1H), 11.94 (Br, 1H), 9.80 (s, 1H), 8.54 (d, J=8.8 Hz, 1H), 8.32 (d, J=8.8 Hz, 1H), 7.92-8.00 (m, 2H), 7.71-7.74 (m, 1H), 7.55-7.58 (m, 1H), 6.80 (Br, 2H); ¹³C NMR (DMSO-d₆, 100 MHz): δ=172.4, 162.3, 157.0, 156.9, 152.8, 151.5, 147.4, 135.8, 129.6, 128.8, 127.8, 126.8, 118.0, 102.0; LCMS (ESI): [M+H]⁺ calculated C₁₄H₁₁N₅OS: 298.0718 found 298.6015.

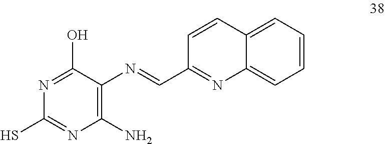 [SMILES: Nc1nc(S)nc(O)c1/N=C/c1ccc2ccccc2n1]

(E)-6-amino-5-((4-hydroxy-3-methoxybenzylidene)amino)-2-mercaptopyrimidin-4-ol (39): Pale Yellow solid; Yield: 84%; Melting Point: 228-230° C.; ¹H NMR (DMSO-d₆, 400 MHz): δ=11.91 (s, 1H), 11.75 (Br, 1H), 9.50 (s, 1H), 9.36 (s, 1H), 7.47 (d, J=1.2 Hz, 1H), 7.14-7.17 (m, 1H), 6.76 (d, J=7.6 Hz, 1H), 6.60 (Br, 2H), 3.81 (s, 3H); ¹³C NMR (DMSO-d₆, 100 MHz): δ=171.4, 157.0, 152.9, 151.5, 148.1, 147.9, 130.0, 126.0, 115.2, 110.6, 102.1, 55.7; LCMS (ESI): [M+H]⁺ calculated C₁₂H₁₂N₄O₃S: 293.0664 found 293.5914.

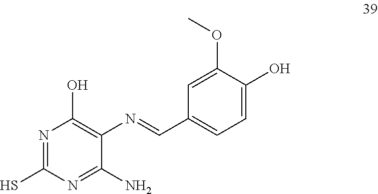 [SMILES: COc1cc(/C=N/c2c(N)nc(S)nc2O)ccc1O]

(E)-6-amino-5-((5-bromo-2-hydroxybenzylidene)amino)-2-mercaptopyrimidin-4-ol (40): Brown solid; Yield: 90%; Melting Point: 254-256° C.; ¹H NMR (DMSO-d₆, 400 MHZ): δ=11.96 (Br, 2H), 10.87 (s, 1H), 9.76 (d, J=6.4 Hz, 1H), 8.04 (d, J=3.6 Hz, 1H), 7.29-7.32 (m, 1H), 6.78-6.82 (m, 1H), 6.72 (Br, 2H); ¹³C NMR (DMSO-d₆, 100 MHz): δ=171.8, 156.9, 156.6, 151.7, 150.0, 133.0, 129.8, 125.3, 118.2, 110.7, 102.2; LCMS (ESI): [M+H]⁺ calculated C₁₁H₉BrN₄O₂S: 343.0609 found 343.3660.

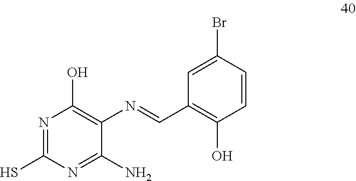 [SMILES: Nc1nc(S)nc(O)c1/N=C/c1cc(Br)ccc1O]

(E)-6-amino-5-((3,5-difluoro-4-hydroxybenzylidene)amino)-2-mercaptopyrimidin-4-ol (41): Pale yellow solid; Yield: 88%; Melting Point: 262-264° C.; ¹H NMR (DMSO-d₆, 400 MHz): δ=11.91 (s, 1H), 11.85 (Br, 1H), 10.50 (s, 1H), 9.36 (s, 1H), 7.61 (s, 1H), 7.59 (s, 1H), 6.50 (Br, 2H); LCMS (ESI): [M+H]⁺ calculated C₁₁H₈F₂N₄O₂S: 299.5555 found 299.0370.

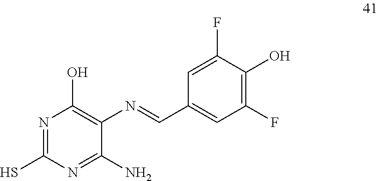 [SMILES: Nc1nc(S)nc(O)c1/N=C/c1cc(F)c(O)c(F)c1]

### Example 2

### Inhibition of DNA Ligase IV Activity

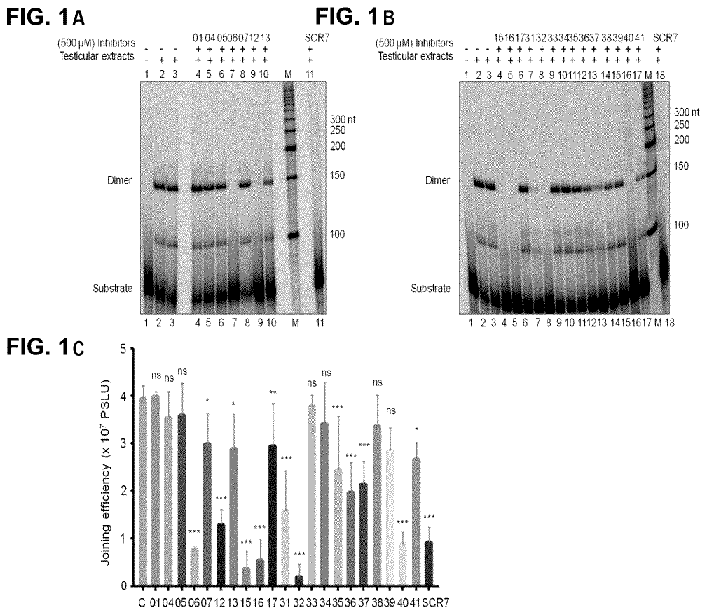

The compounds of the present invention were tested for their effects on inhibition of DNA end joining reactions using rat tissue extracts and purified DNA Ligase IV/XRCC4. Rat testicular extracts were prepared from testes of 4-6 week old male Wistar rats, Rattus norvegicus using ammonium sulphate precipitation method. To determine end joining of cell-free extracts, radiolabeled double-stranded (ds) oligomeric DNA substrate was incubated with rat testicular extract at 25° C. in suitable NHEJ buffer. Extracts were incubated for 30 min with the compounds of Formula I (01-41, 500 μM) as prepared in Example 1, and then radioactive DNA substrate was added to the reaction mixture and incubated for 1 h at 25° C. Reactions were stopped using EDTA, and products were purified using phenol: chloroform extraction and loaded on denaturing polyacrylamide gel. An equivalent amount of DMSO was added to control reactions, whileSCR7 was used as positive control (FIGS. 1A, B and C).It can be understood that the compounds 06, 12, 15, 16, 31, 32 and 40 showed inhibition of end joining (FIG. 1A-C) since in all these cases no joined product was observed after incubation of extract with the compounds. Joining efficiency was calculated using the intensity of product bands (150 bp or higher) in individual lanes in Multi Gauge software as compared to control (lanes 2,3). Lane 1 denotes radioactive substrate in absence of protein or inhibitor, while M denotes radiolabeled 50 bp DNA ladder.

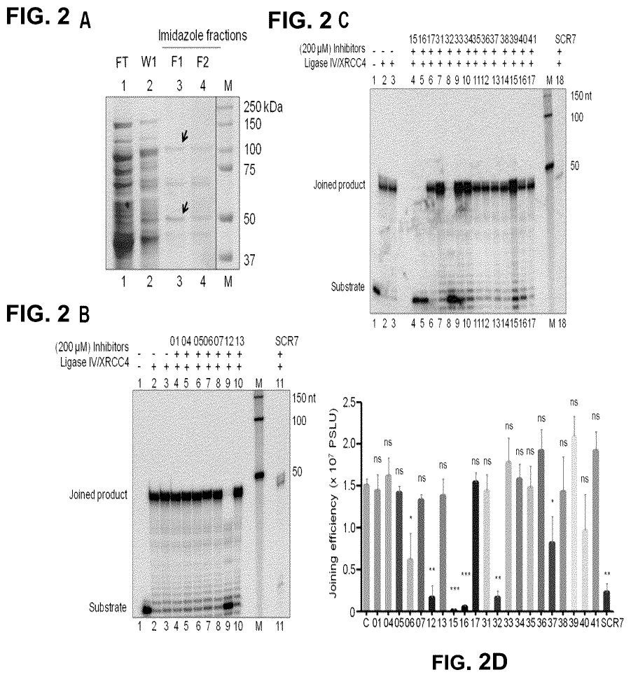

The compounds of Formula I were evaluated for their specific inhibition activity towards enzyme DNA Ligase IV. DNA Ligase IV/XRCC4 was purified from bacteria using Ni-NTA column (FIG. 2A), and used for evaluation of inhibition of end joining of the compounds (01-41) at 200 μM (FIGS. 2B, C and D). The compounds were incubated with purified Ligase IV/XRCC4 in suitable NHEJ buffer at 25° C. for 30 min, followed by addition of radiolabeled DNA, and further incubation for 1 h at 25° C. Products were purified using phenol: chloroform and run on denaturing PAGE. The compounds 12, 15, 16 and 32 showed significant inhibition of end joining as compared with control (FIGS. 2B, C and D).

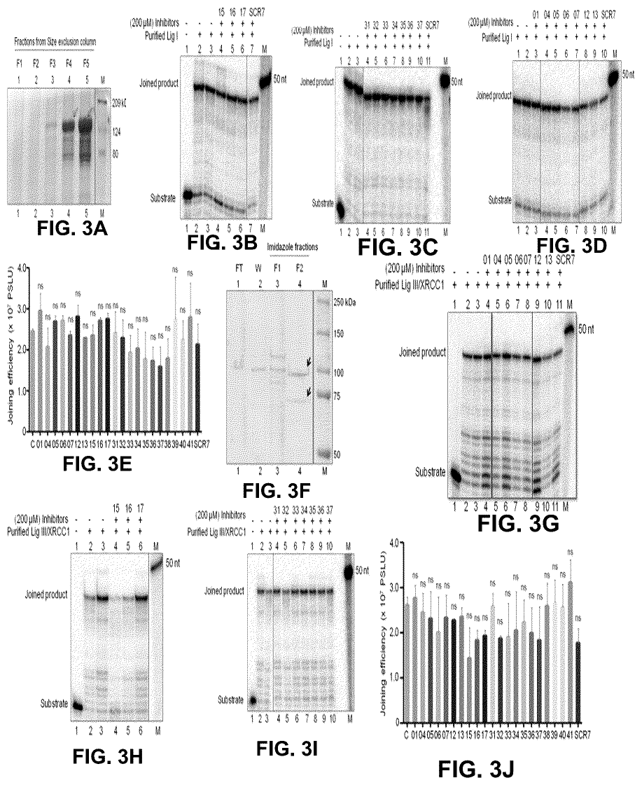

The compounds of the present invention were analysed for inhibition of end joining with purified Ligase I (FIG. 3A) and Ligase III/XRCC1 (FIG. 3F). The compounds were incubated with either purified Ligase I (FIG. 3B-E) or with Ligase III/XRCC1 (FIGS. 3G-J) in suitable NHEJ buffer at 25° C. for 30 min, followed by addition of radiolabeled DNA, and further incubation for 1 h at 25° C. Products were purified using phenol: chloroform, and run on denaturing PAGE. At 200 μM, the compounds of the present disclosure did not show any significant inhibition of Ligase I (FIGS. 3B,C, D and E) and III (FIGS. 3G, H, I and J) mediated joining. Therefore, it can be understood that the compounds of the present invention exhibits selective inhibition activity towards DNA Ligase IV enzyme and had no effect on other DNA Ligases.

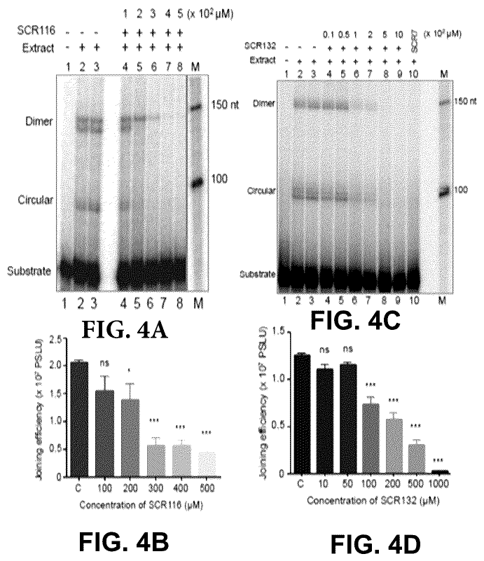

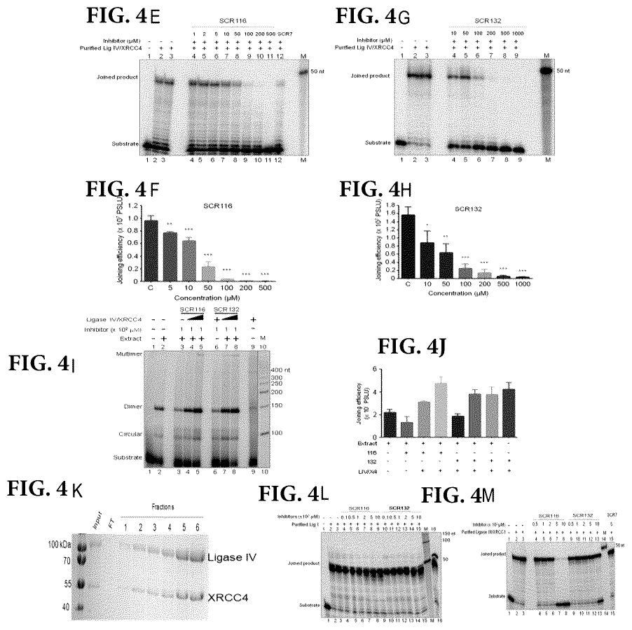

Based on the above results, it was inferred that the compounds SCR116 and SCR 132 showed significant DNA Ligase IV enzyme inhibition. Effects of these compounds on NHEJ and Ligase IV mediated joining were evaluated (FIG. 4A-K). The reactions were terminated by EDTA (10 mM), products were purified using phenol/chloroform, precipitated with chilled ethanol and glycogen, and resolved on 8% denaturing PAGE. The gel was dried, exposed, and signal was detected using phosphor Imager FLA9000 (Fuji, Japan), and quantified in MultiGauge software expressed as Photo Stimulated Luminescence Units (PSLU). Inhibition of end joining was reported at 200 μM and 100 μM, respectively for SCR116 and SCR132 (FIGS. 4A, B, C and D), with >50% inhibition at 200 and 100 μM concentrations, respectively. Compounds SCR116 and SCR132 showed complete inhibition of end joining at 200 μM. A dose titration experiment with the compounds at increasing concentrations of 1, 2, 5, 10, 50, 100, 200, 500 M for the former and 10, 50, 100, 200, 500 μM for the latter (FIGS. 4E, F, G and H) with Ligase IV in suitable NHEJ buffer and radioactively labeled DNA substrate revealed inhibition of end joining from 5 μM for SCR116 and 10 μM for SCR132. End joining efficiency decreased continuously in a dose-dependent manner ((FIGS. 4E-H). Both the compounds SCR116 and SCR132, greater than 50% inhibition was observed at 50 μM concentration (FIGS. 4E-H), which was up to 4-fold lower than that of SCR7. Further, when Ligase IV was added back to reconstitute the reaction after inhibition of testicular extract with the compounds, SCR116 and SCR132, joining efficiency was restored, suggesting Ligase IV dependent effects (FIGS. 4I-J). These compounds SCR116 and SCR132 showed no inhibition even at highest concentration of 1000 μM with Ligase I. With Ligase III/XRCC1, SCR132 showed no significant inhibition, although SCR116 inhibited end joining at highest concentrations of 500 and 1000 μM, which could be due to similarities in domain structures of DNA Ligases III and IV. FIG. 4K depict the purification profile of Ligase IV and XRCC4. Further FIGS. 4L-M represent inhibition of end joining catalysed by (FIG. 4L) purified Ligase III/XRCCI in presence of SCR116 and SCR132 (50, 100, 200, 500, 1000 μM) and (FIG. 4M) by purified Ligase I in presence of increasing concentrations of SCR116 and SCR132 (10, 50, 100, 200, 500, 1000 μM). These results confirmed that SCR116 showed inhibition from 5 μM, SCR132 inhibited end joining from 10 μM, the efficiency of which increased in a dose-dependent manner. Importantly, >50% inhibition was observed at 50 μM concentration for both the compounds. Also, SCR116 and SCR132 did not show inhibition of end joining catalyzed by purified Ligase III and Ligase I.

### Example 3

Treatment of Cancer Cell Lines with the Compounds of the Present Disclosure

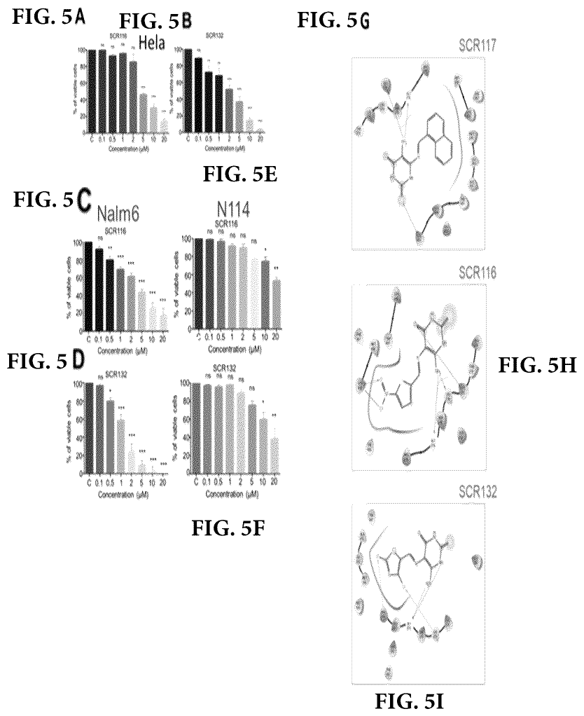

The compounds of the present invention were screened initially in leukemic and cervical cancer cell lines (Table 1). IC₅₀ values for the selected compounds in Reh cells were 2.3 μM (SCR116) and 690 nM (SCR132) at 48 h, which is >100 fold and >250 fold respectively than that of SCR7. In cervical cancer cell line HeLa, IC₅₀ was 5 and 2 μM, respectively for SCR116 and SCR132 (FIG. 5A-B). It was inferred that the compounds SCR116 and SCR132 had IC₅₀ values 300 folds lower than that of SCR7 in different cancer cell lines and SCR132 showed cancer cell death at nanomolar concentrations.

Ligase IV specificity of the compounds of the present invention inside the cells were also determined using Ligase IV knock out (KO) cell line N114 compared to the wild type Nalm6 (Table 2, FIGS. 5C, D, E and F). SCR7 showed 5 fold increased IC₅₀ (>250 μM) in N114, while SCR7-pyrazine showed nonspecific effects in KO in absence of Ligase IV (Vartak et al., The FEBS Journal 285, 3959-3976,2018). Cells were treated with increasing concentrations of SCR116 and SCR132 for 48 h, and IC₅₀ values were obtained at 14.09 and 10.13 μM, respectively, suggesting 4-5 fold reduction. Thus, in absence of target protein Ligase IV the compounds of the present disclosure did not show cancer cell death, suggesting Ligase IV specificity in vivo. Table 1 showing IC₅₀ of SCR7 derivatives in cancer cell lines Nalm6 and HeLa. IC₅₀ was calculated using GraphPad Prism software from the percentage of viable cells following treatment with the compounds at 48 h. Table 2 showing IC₅₀ of SCR116 and SCR132 in a panel of 16 cancer cell lines.

Evaluation of IC₅₀ of SCR7 derivatives in Nalm6 and HeLa cell lines and molecular docking studies was carried out with DNA Ligase IV (FIGS. 5G-I). Table 3 illustrates glide and dock scores for the derivatives docked to Ligase IV crystal structure (3W5O).

In silico analysis revealed SCR116 and SCR132 were the top two compounds that showed promising effects. When SCR116 was docked with Ligase IV, the electron withdrawing group —NO2 showed interaction with Lys432, —NH₂ with Gly276 and Leu274, and —CO with Lys273 on Ligase IV. Electron withdrawing —Cl groups of SCR132 interacted with Arg278 and Leu274, while —NH₂ and —NH bound to Gly276 in the binding pocket of Ligase IV.

TABLE 1
IC₅₀ (μM) in cancer cell lines

| Compounds | Nalm6 | HeLa |
|---|---|---|
| 01 | 23.30 | 17.72 |
| 04 | 29.69 | 94.95 |
| 05 | 18.22 | 11.90 |
| 06 | 19.35 | 17.46 |
| 07 | 26.57 | 28.16 |
| 12 | 29.51 | 48.47 |
| 13 | 25.55 | 133.4 |
| 15 | 29.81 | 79.77 |
| 16 | 3.44 | 5.92 |
| 17 | 29.72 | 29.52 |
| 31 | 4.04 | 6.95 |
| 32 | 1.79 | 2.08 |
| 33 | 2.24 | 20.83 |
| 34 | 0.95 | 41.41 |
| 35 | 6.87 | 18.40 |
| 36 | 2.88 | 27.18 |
| 37 | 15.23 | 47.75 |
| 38 | 25.51 | 45.77 |
| 39 | 33.61 | 43.16 |
| 40 | 12.55 | 18.29 |
| 41 | 28.76 | 29.17 |

TABLE 2
IC₅₀ (μM) in cancer cell lines

| Cell line | SCR116 | SCR132 | SCR7 |
|---|---|---|---|
| Nalm6 | 3.44 | 1.79 | 50 |
| Reh | 2.34 | 0.690 | >250 |
| HeLa | 5.92 | 2.083 | 44 |
| Supt1 | 1.32 | 0.734 | 108.9 |
| Raji | 4.37 | 1.587 | >250 |
| CEM | 5.77 | 3.83 | >200 |
| SUDHL8 | 0.685 | 0.810 | 100 |
| N114 | 14.09 | 10.13 | >250 |
| HCT116 | 5.2 | 2.0 | 302.3 |
| LN229 | 5.6 | 7.7 | 59.9 |
| U118 | 5.6 | 4.1 | 150.9 |
| EAC | 16.49 | 14.79 | 131.8 |
| L1210 | 3.75 | 0.879 | 370.3 |
| Molt4 | 4.89 | 1.88 | 182.42 |
| Molt3 | 3.1 | 0.795 | 79.1 |
| MCF7 | 10 | 5 | 40 |

TABLE 3

| | Glide score | Dock score |
|---|---|---|
| Compound | (kcal/mol) | (kcal/mol) |
| SCR117 | −6.15 | −6.15 |
| SCR106 | −6.10 | −6.02 |
| SCR140 | −6.24 | −5.98 |
| SCR112 | −5.92 | −5.83 |
| SCR105 | −5.74 | −5.69 |
| SCR116 | −5.66 | −5.58 |
| SCR107 | −6.71 | −5.28 |
| SCR137 | −5.28 | −5.21 |
| SCR132 | −5.10 | −4.99 |
| SCR134 | −5.05 | −4.97 |
| SCR104 | −6.15 | −4.96 |
| SCR113 | −4.95 | −4.89 |
| SCR135 | −4.92 | −4.84 |
| SCR138 | −4.75 | −4.63 |
| SCR131 | −5.19 | −4.61 |
| SCR133 | −4.88 | −4.59 |
| SCR141 | −4.48 | −4.42 |
| SCR115 | −4.52 | −4.38 |
| SCR101 | −4.36 | −4.28 |
| SCR136 | −4.18 | −4.17 |
| SCR139 | −4.17 | −4.15 |

### Example 4

### Evaluation of V(D)J Recombination Efficiency

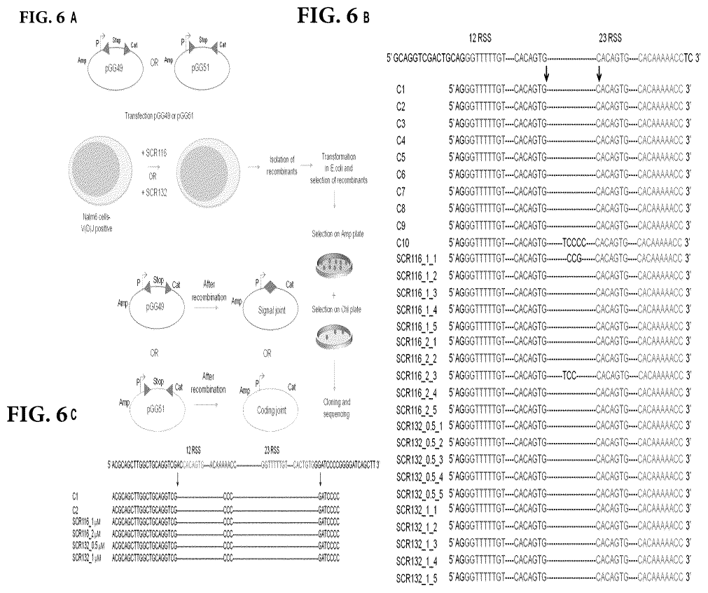

Evaluation of efficiency of V(D)J recombination following treatment with SCR116 and SCR132 was carried out as depicted in FIGS. 6(A-C). Tables 4 and 5 summarize the effect of SCR116 (1 and 2 μM) and SCR132 (0.5 and 1 μM) on V(D)J recombination in Nalm6 using pGG49 and pGG51, respectively. Results 5 showed that, in pGG49, 5.2 (1 μM) and 20 (2 μM) fold reduction in recombination efficiency was observed with SCR116, while 9.3 (0.5 μM) and 31.1 (1 μM) fold reduction was seen with SCR132. Moreover, a greater reduction in V(D)J recombination efficiency was observed in the case of pGG51 with SCR116 (9 and 54-fold) and SCR132 (50 and 106-fold).

TABLE 4

| | | | | Recombination | Fold reduction |
|---|---|---|---|---|---|
| SI | | Amp (A) | CA | efficiency | in recombination |
| No. | pGG49 | colonies | colonies | (CA/A) ×100 | efficiency |
| 1. | Vehicle | 115940 | 312 | 0.260 | 1.0 |
| | control | | | | |
| 2. | SCR116 | 100880 | 65 | 0.050 | 5.2 |
| | 1 μM | | | | |
| 3. | SCR116 | 106320 | 14 | 0.013 | 20.0 |
| | 2 μM | | | | |
| 4. | Vehicle | 84160 | 240 | 0.280 | 1.0 |
| | control | | | | |
| 5. | SCR132 | 122880 | 42 | 0.030 | 9.3 |
| | 0.5 μM | | | | |
| 6. | SCR132 | 101207 | 10 | 0.009 | 31.1 |
| | 1 μM | | | | |

TABLE 5

| | | | | Recombination | Fold reduction |
|---|---|---|---|---|---|
| SI | | Amp (A) | CA | efficiency | in recombination |
| No. | pGG51 | colonies | colonies | (CA/A) ×100 | efficiency |
| 1. | Vehicle | 127840 | 348 | 0.270 | 1.0 |
| | control | | | | |
| 2. | SCR116 | 113880 | 40 | 0.030 | 9.0 |
| | 1 μM | | | | |
| 3. | SCR116 | 139540 | 8 | 0.005 | 54.0 |
| | 2 μM | | | | |
| 4. | Vehicle | 122540 | 400 | 0.320 | 1.0 |
| | control | | | | |
| 5. | SCR132 | 154600 | 10 | 0.006 | 50.0 |
| | 0.5 μM | | | | |
| 6. | SCR132 | 151920 | 5 | 0.003 | 106.6 |
| | 1 μM | | | | |

Compounds Used in Combination with Radiation and Chemotherapeutic Agents

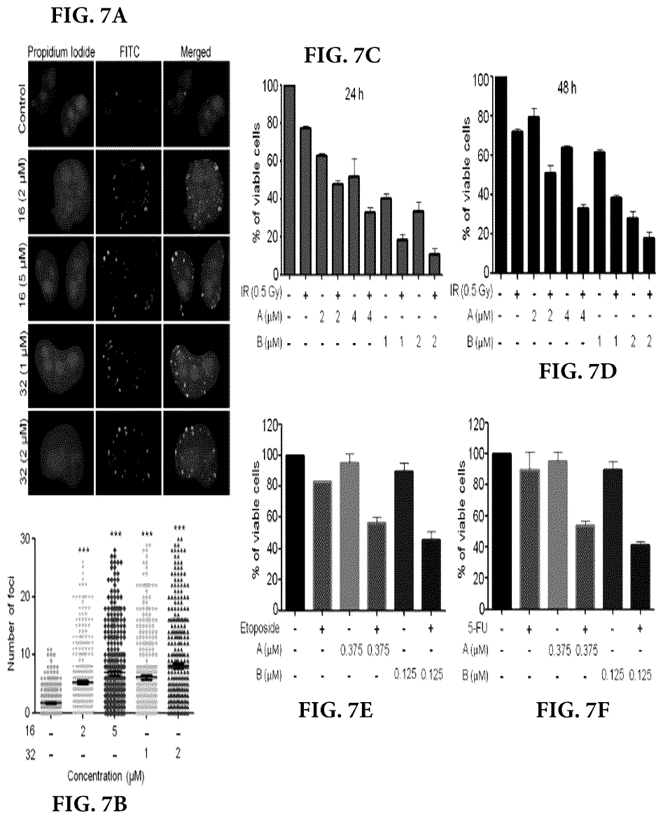

In order to evaluate the effect of the inhibitors (SCR116 & SCR132) on DSB generation in cancer cells, the cells were treated with the compounds alone or alongside radiation, and an increase in DNA damage marker 53BP1 was observed (FIGS. 7A-B). Increased cytotoxicity was also observed with SCR116 and SCR132 when combined with 0.5 and 1 Gy γ-radiation, or chemotherapeutic agents (5-FU and etoposide) (FIGS. 7C-F).

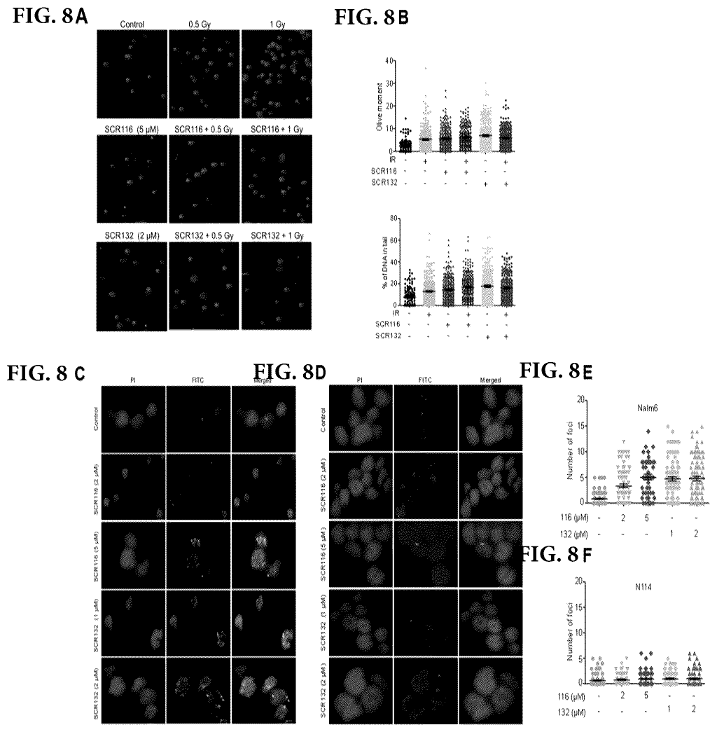

FIG. 8(A-B) depicts accumulation of DSBs inside cells following treatment with the compounds. A. Representative images for HeLa cells subjected to neutral comet assay following treatment with SCR116 and SCR132 for 24 h. B. Scatter plot depicting quantification of percentage of DNA in tail (bottom) and olive moment (top) in HeLa cells treated with SCR116 (5 μM) and SCR132 (2 μM) in presence of radiation (0.5 Gy).Results revealed increased amount of DNA in the tail of the comet in treated cases as compared to control. FIG. 8C, D are representative images for Nalm6 cells (C) and N114 cells (D) following treatment with SCR116 and SCR132 for 24 h. and FIG. 8E, F are scatter plot depicting quantification of percentage of 53BP1 foci treated with SCR116 (2, 5 μM) and SCR132 (1, 2 μM) in Nalm6 and N114. Results revealed that there was a significant increase in 53BP1 foci in Nalm6 cells following SCR116 and SCR132 treatment. However, no significant increase in the number of 53BP1 foci was observed in N114 cells following treatment with SCR116 and SCR132.

### Example 5

### Upregulation of Apoptosis

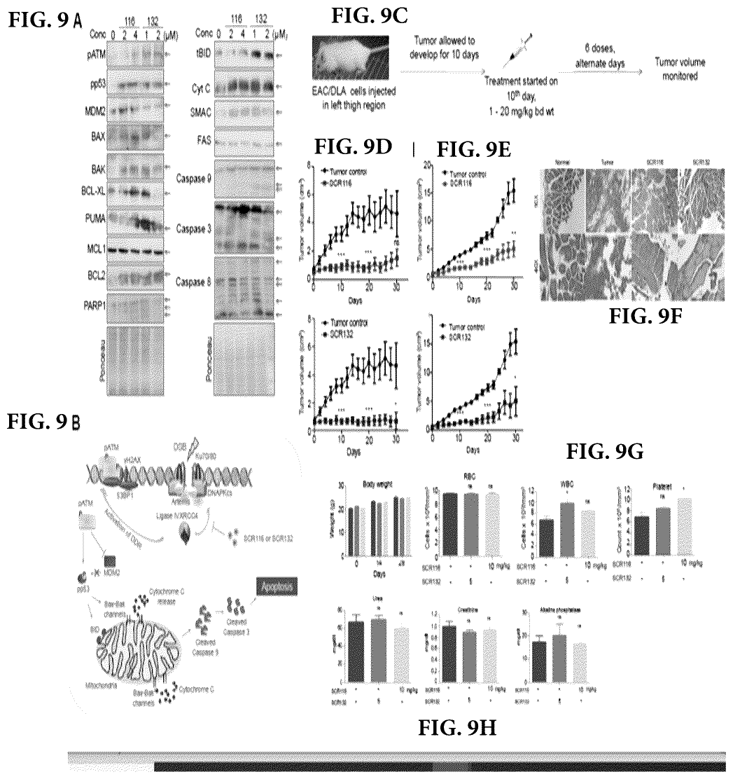

To investigate the signaling pathways involved in apoptosis post treatment with the compounds in leukemic cell lines Nalm6 and Reh, western blot analysis was performed. A concentration dependent increase in pATM, pp53, CYTOCHROME C, BAX, BAK and tBID was observed, suggesting activation of intrinsic apoptotic pathway (FIG. 9A). Although BCL2 and MCL1 remained constant, cleaved PARP1, cleaved Caspase 3 and cleaved Caspase 9 was observed to increase in both cell extracts. A likely involvement of Caspase 8 in extrinsic pathway was observed, although expression of FAS and SMAC-DIABLO proteins remained.

Based on the above results, the working model for the compounds were determined. SCR116 and SCR132 bound to DBD Ligase IV and inhibit NHEJ. The accumulating breaks inside cell could trigger DNA damage response, activation of apoptotic pathway, opening of Bax-Bak channels, release of Cytochrome C and cleavage of Caspases. (FIG. 9B).

### Anti-Cancer Activity of the Compounds of the Present Disclosure

The compounds of the present disclosure were evaluated for the anti-cancer efficacy starting from lower concentrations using Mice allograft model. Mice allograft model was developed by injecting Erlich Ascites Carcinoma (EAC) cell lines in the thigh region of the mice followed by treatment with the compounds via intraperitoneal and oral routes on alternate days (FIG. 9C). The compounds showed inhibition of tumor progression at concentrations 10 mg/kg for SCR116 and 5 mg/kg for SCR132 when treated in EAC tumor model via intraperitoneal route (FIGS. 9D-E). Histological evaluation of mice thigh regions in normal, tumor control and tumor treated samples revealed infiltrated tumor cells in tumor control, and elimination of most of these infiltrated cells in treatment conditions with restoration of muscle architecture (FIG. 9F). Preliminary analysis of toxicity after 14 days in the normal mice post administration of 6 doses of the compounds via intraperitoneal route showed no significant changes in body weight or blood parameters between control and treatment groups (FIGS. 9G,H).

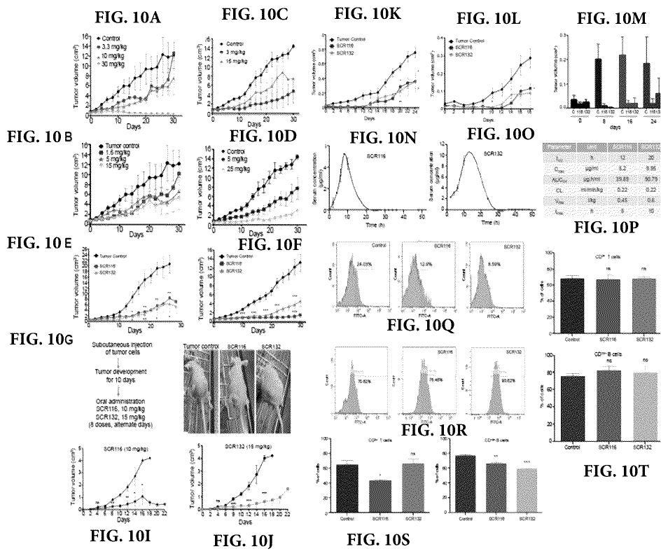

The effect of oral dose of the compounds of the present disclosure on mice Erlich Ascites Carcinoma (EAC) and Dalton's Lymphoma (DLA) tumors were also evaluated (FIGS. 10A-F). In the oral dose (8 doses, alternate days) in EAC tumor bearing mice, the animals showed reduced tumor load at 10 and 30 mg/kg (for SCR116) and 5 and 15 mg/kg (for SCR132) (FIGS. 10A,B). Oral dose of 15 and 25 mg/kg of SCR116 and SCR132 in DLA tumor bearing mice also showed reduced tumor burden throughout the pilot experiment (FIGS. 10C,D). Based on these studies, the selected oral dose of 10 and 15 mg/kg for SCR116 and SCR132 respectively were evaluated in EAC and DLA with significant reduction in tumor size (FIGS. 10E,F).

Effect of the compounds was further evaluated in mouse xenograft model generated using Ligase IV high human leukemic cell line, Molt4. Oral doses of SCR116 and SCR132 at 10 mg/kg and 15 mg/kg respectively were administered on alternate days. Both the compounds effectively reduced the tumor volume (FIGS. 10G, H, I and J). While all the tumor control animals died by day 18, those in the treatment groups survived the entire experimental period (FIG. 10H-J), suggesting improved survivability. Effects of the compounds were also evaluated in oral cancer (SCC084 and SCC131) and hepatocellular cancer (HCT116) (FIG. 10K-M), with significant reduction in tumor volume post treatment.

### Improved Bioavailability

The compounds of the present disclosure showed promising anti-cancer effects in allograft and xenograft models upon oral administration in mice, fraction of the compounds reaching systemic circulation post metabolism was evaluated using HPLC analysis. SCR7 showed bioavailability of 114 μg/ml and a t_{1/2} of 1 h when administered intraperitoneally at 20 mg/kg body weight of mice. The bioavailability of the compounds of the present disclosure were analysed in mice serum at different time points (0.5, 1, 2, 4, 6, 8, 10, 12, 24 and 48 h) by HPLC at 232 nm wavelength (FIGS. 10N-P). Both the inhibitors reached circulation by 1 h in mice and continued to increase till 10 h (FIG. 10N-P). SCR132 became stabilized between 4 h and 6 h in the serum, while SCR116 continued to increase till 10 h, suggesting differential release of compounds into circulation. The area under the curve (AUC) at 10 h was highest for the compounds (FIGS. 10N-P), suggesting better bioavailability than SCR7 even at 2-4 fold lower concentrations.

The effects of the compounds on V(D)J recombination in mice were also evaluated since NHEJ is involved in the last steps of the recombination process for repair of the double-strand breaks. V(D)J recombination is a process involved in generating antibody diversity in living organisms during the process of development of B and T lymphocytes. Animals were administered with the compounds orally for 3 consecutive days, and analysed for total B and T cells by flow cytometry. The treated animals showed reduction in numbers of B cells, without much difference in T cells (FIGS. 10Q-R), suggesting effects on in vivo V(D)J recombination. This was followed by a recovery experiment, where the numbers of B and T cells were evaluated for 21 days post treatment. Since the numbers were restored after the recovery period, the compounds showed transient effects in vivo (FIGS. 10S-T).

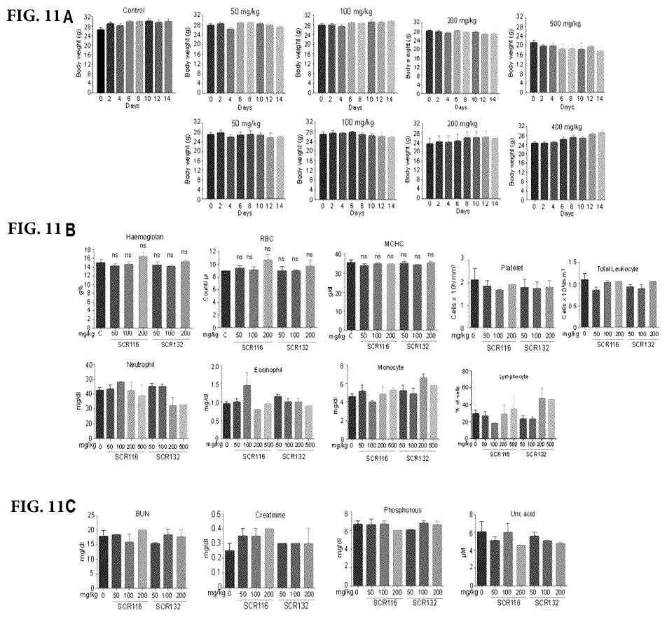

The toxic effects of the compounds of the present disclosure were determined by oral administration at several fold higher concentrations in mice than intended to exert their effects. According to the CDSCO guidelines for drug development and administration, compounds were orally administered as a single dose of 50, 100, 200, 500, 1000, 1500 and 2000 mg/kg body weight (FIG. 11A-C) and monitored for 14 days for any changes in body weight, food habits or behavior. No significant change was observed in body weight, or RBC, WBC and platelet counts between control and animals treated with 50, 100 and 200 mg/kg body weight. Thus, even 20 times high dose (200 mg/kg) did not cause any toxicity to the animals (FIGS. 11A-C).

Further, kidney-liver function tests such as Blood urea nitrogen (BUN), creatinine, uric acid content, ALP, SGPT, SGOT, etc. were also performed to determine the effect of the compounds on kidney and liver conditions. When compared between treated and control groups none of the kidney-liver function parameters showed fluctuations in a dose-dependent manner (FIG. 11C). Till 200 mg/kg concentration the compounds showed no toxic effects, which was >10 fold higher dose intended for use. Therefore, the compounds exhibited no preclinical toxic effects in mice when administered at several fold higher concentrations.

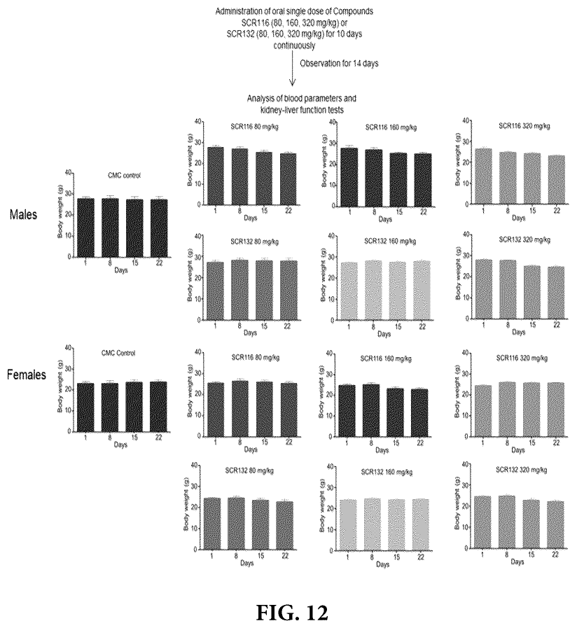

FIG. 12 depicts 10 days dose ranging toxicity studies of SCR116 and SCR132 (A) Body weight analyses of mice on 1, 8, 15 and 22 days after oral administration of low, medium and high concentrations of SCR116 and SCR132 (80, 160 and 320 mg/kg b.wt. respectively) in male. Mice treated with methyl cellulose served as vehicle control; and (B) Body weight analyses of mice on 1, 8, 15 and 22 days after oral administration of SCR116 and SCR132 (80, 160 and 320 mg/kg b.wt. respectively) in female. The results confirmed no significant change in body weight.

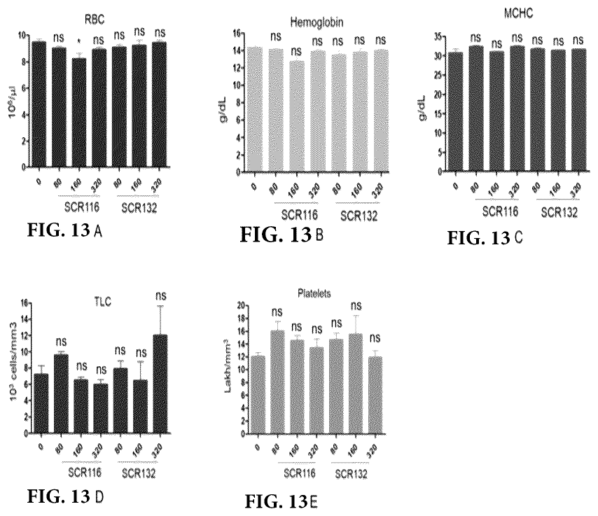

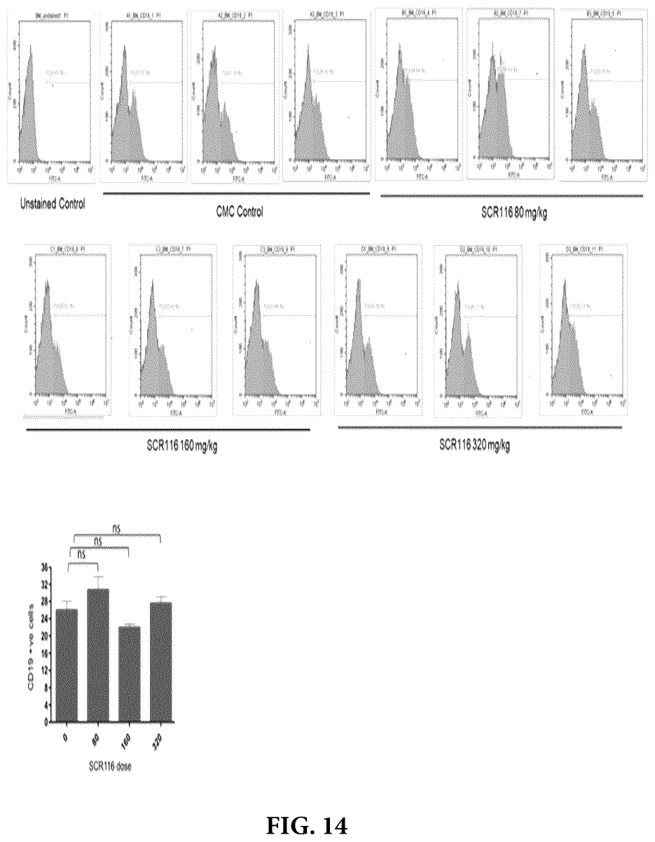

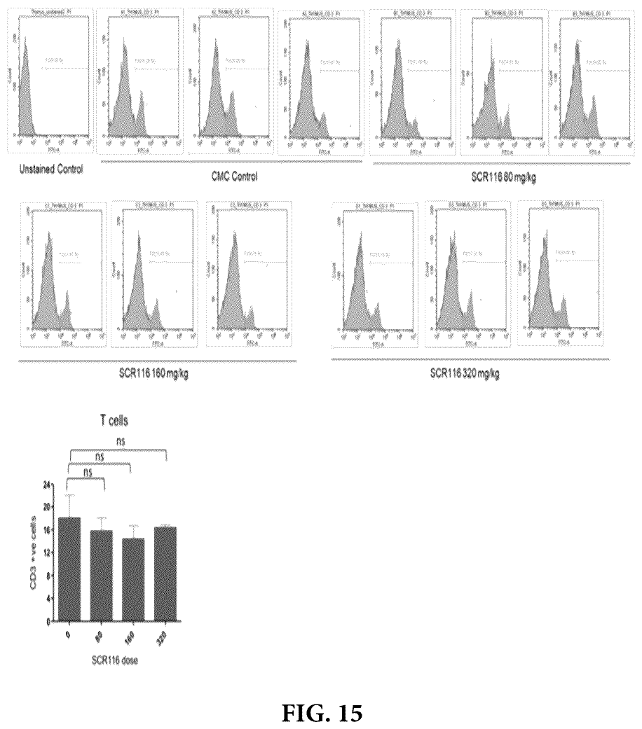

FIG. 13 depicts the blood parameters analysis of Swiss albino mice after 10 days of dose-ranging study after treating with SCR116 and SCR132. A. RBC (n=3). B. Hemoglobin (n=3). C. MCHC (n=3). D. Total leukocyte count (n=3). E. Platelet count (n=3) (ns: not significant, *p<0.05: **p<0.005). The studies showed that there was no significant difference in hemoglobin, MCHC, total leukocyte count and platelets. However, there was a significant difference in RBC count in one of the groups when low doses of SCR116 was used, but the values obtained were still within the normal range only. Similarly FIGS. 14(A-B) and FIGS. 15(A-B) depicted that there were no significant changes observed in production of B cells and T cells after SCR116 treatment, respectively.

### Advantages of the Present Disclosure

The present disclosure provides mercaptopyrimidine compounds of Formula I that are inhibitors of DNA Ligase IV enzyme and thus the NHEJ pathway. The compounds of the present disclosure inhibits DNA Ligase IV enzyme and does not have any effect on other DNA Ligases. Therefore the compounds of the present disclosure is effective towards treatment of cancer wherein the DNA Ligase IV enzyme expression is higher. The compounds of the present disclosure have low IC₅₀ values in the micromolar to nanomolar range in various cancer cell lines and therefore are highly efficient pharmaceutical compounds. The compounds showed 5 fold better efficacy in inhibition of Ligase IV mediated end joining in biochemical studies. The compounds showed anti-cancer effects in mice tumor models at 5 fold lower doses particularly by oral route. Bioavailability of the compounds are also higher as these compounds reached circulation quickly, and stayed in blood for longer hours. No significant toxic effects is also observed. The compounds can also be used in combination therapy which provides higher efficacy in treating the wide range of cancer cells. These compounds are also promising for targeted therapy in the treatment of cancers.

1. A compound of Formula I, 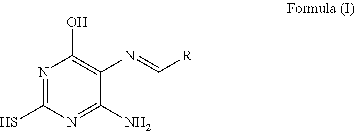
its stereoisomers, intermediates, and pharmaceutically acceptable salts thereof,
wherein R is selected from a group consisting of substituted monocyclic C₆₋₁₀ aryl, C₁₋₁₂ alkyl, bicyclic C₈₋₁₂ aryl, C₃₋₁₀heteroaryl, C₃₋₁₀ cycloalkyl, and C₃₋₁₀heterocyclyl; wherein bicyclic C₈₋₁₂ aryl, C₁₋₁₂ alkyl, C₃₋₁₀heteroaryl, C₃₋₁₀ cycloalkyl, and C₃₋₁₀heterocyclyl are optionally substituted.

2. The compound as claimed in claim 1, its stereoisomers, intermediates, and pharmaceutically acceptable salts thereof, wherein R is monocyclic C₆₋₁₀ aryl substituted with one or more substituents selected from the group consisting of hydroxyl, —R′O(O)CR″, nitro, halogen, cyano, amino, aminoC₁₋₆alkyl, and C₁₋₆alkoxy; and R′ and R″ is independently selected from C₁₋₆alkyl.

3. The compound as claimed in claim 1, its stereoisomers, intermediates, and pharmaceutically acceptable salts thereof, wherein R is selected from bicyclic C₈₋₁₂ aryl, C₁₋₁₂ alkyl, C₃₋₁₀ cycloalkyl, C₃₋₁₀ heteroaryl, or C₃₋₁₀heterocyclyl,
wherein C₃₋₁₀ heteroaryl and C₃₋₁₀heterocyclyl has one to three hetero atoms selected from N, O and S;
wherein bicyclic C₈₋₁₂ aryl, C₁₋₁₂ alkyl, C₃₋₁₀ cycloalkyl, C₃₋₁₀ heteroaryl, or C₃₋₁₀heterocyclyl is optionally substituted with one or more substituents selected from the group consisting of hydroxyl, —R′O(O)CR″, nitro, halogen, cyano, amino, aminoC₁₋₆alkyl, and C₁₋₆alkoxy and
R′ and R″ is independently selected from C₁₋₆alkyl.

4. The compound as claimed in claim 1, its stereoisomers, intermediates, and pharmaceutically acceptable salts thereof, wherein R is selected from substituted monocyclic C₆₋₁₀ aryl, bicyclic C₈₋₁₂ aryl, or C₃₋₁₀ heteroaryl,
wherein monocyclic C₆₋₁₀ aryl is substituted with one or more substituents selected from hydroxyl, halogen, aminoC₁₋₆ alkyl, cyano, and C₁₋₆ alkoxy; and
bicyclic C₈₋₁₂ aryl, and C₃₋₁₀ heteroaryl is optionally substituted with one or more substituents selected from —CH₂O(O)CCH₃, nitro, halogen, and cyano.

5. The compound as claimed in claim 1, its stereoisomers, intermediates, and pharmaceutically acceptable salts thereof, wherein the compound is selected from the group consisting of:
i. (E)-6-amino-2-mercapto-5-(((5-nitrothiophen-2-yl)methylene)amino)pyrimidin-4-ol;
ii. (E)-6-amino-5-(((2,4-dichlorothiazol-5-yl)methylene)amino)-2-mercaptopyrimidin-4-ol;
iii. (E)-6-amino-2-mercapto-5-((thiophen-2-ylmethylene)amino)pyrimidin-4-ol;
iv. (E)-5-(((1H-imidazol-2-yl)methylene)amino)-6-amino-2-mercaptopyrimidin-4-ol;
v. 6-amino-5-{[(E)-isoquinolin-4-ylmethylidene]amino}-2-mercaptopyrimidin-4-ol;
vi. (E)-6-amino-5-((2-hydroxybenzylidene)amino)-2-mercaptopyrimidin-4-ol;
vii. (E)-6-amino-2-mercapto-5-((pyridin-3-ylmethylene)amino)pyrimidin-4-ol;
viii. (E)-5-(((1H-pyrrol-2-yl)methylene)amino)-6-amino-2-mercaptopyrimidin-4-ol;
ix. (E)-6-amino-2-mercapto-5-((thiazol-2-ylmethylene)amino)pyrimidin-4-ol;
x. (E)-(5-(((4-amino-6-hydroxy-2-mercaptopyrimidin-5-yl)imino)methyl)furan-2-yl)methylacetate;
xi. (E)-6-amino-2-mercapto-5-((naphthalen-1-ylmethylene)amino)pyrimidin-4-ol;
xii. (E)-6-amino-5-(((2-chloropyridin-4-yl)methylene)amino)-2-mercaptopyrimidin-4-ol;
xiii. (E)-6-amino-5-(((3-fluoropyridin-4-yl)methylene)amino)-2-mercaptopyrimidin-4-ol;
xiv. (E)-5-(((4-amino-6-hydroxy-2-mercaptopyrimidin-5-yl)imino)methyl)thiophene-2-carbonitrile;
xv. (E)-6-amino-5-(((5-bromothiophen-2-yl)methylene)amino)-2-mercaptopyrimidin-4-ol;
xvi. (E)-4-(((4-amino-6-hydroxy-2-mercaptopyrimidin-5yl)imino)methyl)benzonitrile;
xvii. (E)-6-amino-5-((4-(dimethylamino)benzylidene)amino)-2-mercaptopyrimidin-4-ol;
xviii. (E)-6-amino-2-mercapto-5-((quinolin-2-ylmethylene)amino)pyrimidin-4-ol;
xix. (E)-6-amino-5-((4-hydroxy-3-methoxybenzylidene)amino)-2-mercaptopyrimidin-4-ol;
xx. (E)-6-amino-5-((5-bromo-2-hydroxybenzylidene)amino)-2-mercaptopyrimidin-4-ol; and
xxi. (E)-6-amino-5-((3,5-difluoro-4-hydroxybenzylidene)amino)-2-mercaptopyrimidin-4-ol.

6. The compound as claimed in claim 1, its stereoisomers, intermediates, and pharmaceutically acceptable salts thereof, wherein the compound inhibit DNA Ligase IV enzyme activity and nonhomologous end joining (NHEJ).

7. The compound as claimed in claim 1, its stereoisomers, intermediates, and pharmaceutically acceptable salts thereof, for the manufacture of a medicament for treatment of cancer associated with expression of DNA Ligase IV enzyme.

8. (canceled)

9. A process for preparing the compound of Formula I as claimed in claim 1, the process comprising:
reacting 5,6-diamino-2-mercaptopyrimidin-4-ol with a compound of Formula B in the presence of an acid and a solvent under stirring for a time period of 6 to 8 hours to obtain the compound of Formula I, 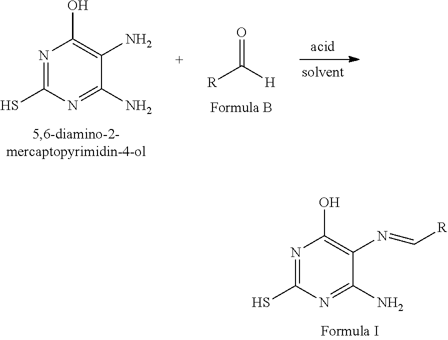
wherein R is selected from a group consisting of substituted monocyclic C₆₋₁₀ aryl, C₁₋₁₂ alkyl, bicyclic C₈₋₁₂ aryl, C₃₋₁₀ heteroaryl, C₃₋₁₀ cycloalkyl, and C₃₋₁₀heterocyclyl.

10. The process as claimed in claim 9, wherein the acid is selected from glacial acetic acid, formic acid, or oxalic acid; and the solvent is dimethyl formamide or dimethyl sulfoxide.

11. A pharmaceutical composition comprising the compound of Formula I as claimed in claim 1, with a pharmaceutically acceptable adjuvant, carrier, or vehicle.

12. The pharmaceutical composition as claimed in claim 11, wherein the pharmaceutical composition is in a form selected from a tablet, capsule, powder, syrup, solution, aerosol or suspension.

13. A pharmaceutical combination comprising the compound of Formula I as claimed in claim 1 with one or more second therapeutic agent.

14. The pharmaceutical combination as claimed in claim 13, wherein the one or more second therapeutic agent is selected from a chemotherapeutic agent, a radiotherapeutic agent, an antiproliferative agent, an antineoplastic agent, DNA-damaging active compounds, or combinations thereof.

15. The pharmaceutical combination as claimed in claim 14, wherein the chemotherapeutic agent is selected from 5-fluorouracil, azacytidine, paclitaxel, bendamustine, etoposide, bleomycin, temozolomide, cisplatin, or AZD2461; and the radiotherapeutic agent is selected from ionizing radiation (γ-rays).

16. A method of treating a cancer in a subject in need thereof, comprising administering an effective amount of the compound of Formula I as claimed in claim 1.

17. The method as claimed in claim 16, wherein the cancer is selected from leukemia, lymphoma, head and neck carcinoma, colon carcinoma, lung cancer, prostate cancer, or glioma.

18. A method for inhibiting DNA Ligase IV enzyme activity with an effective amount of the compound as claimed in claim 1.

19. A method of inhibiting a DNA double-strand break through NHEJ, the method comprising contacting the compound of Formula I as claimed in claim 1 with DNA Ligase IV.

20. The method as claimed in claim 19, wherein the DNA Ligase is DNA Ligase IV and the method is carried out by NHEJ.

21. (canceled)

22. A DNA repair kit comprising the compound as claimed in claim 1.
